(19) United States
(12) Patent Application Publication
(10) Pub. No.: US 2026/0335305 A1
(43) Pub. Date: Oct. 29, 2026

(54) METHODS OF USING ITRACONAZOLE DRY POWDERS

(71) Applicant: Pulmatrix Operating Company, Inc., Framingham, MA (US)

(72) Inventor: Aidan K. CURRAN, Duxbury, MA (US)

(21) Appl. No.: 19/107,206

(22) PCT Filed: Aug. 31, 2023
(86) PCT No.: PCT/US2023/031675

§ 371 Date: Feb. 27, 2025

Related U.S. Application Data
(60) Provisional application No. 63/402,577, filed on Aug. 31, 2022.

(51) Int. Cl. A61K 31/496; A61K 9/00; A61K 9/16; A61K 45/06

(52) U.S. Cl. CPC A61K 31/496; A61K 9/0075; A61K 9/1611; A61K 9/1617; A61K 9/1623; A61K 45/06

(57) ABSTRACT

The present disclosure relates to a method of treating a disease or disorder in a subject for whom oral itraconazole is contraindicated, comprising administering to the respiratory tract of the subject a respirable dry powder comprising itraconazole. The present disclosure also relates to methods of co-administering itraconazole with a second therapeutic agent to a subject in need thereof, wherein the itraconazole is administered as a respirable dry powder to the respiratory tract of the subject, and wherein the second therapeutic agent is a substrate, inducer, and/or inhibitor of an enzyme or receptor that is inhibited by, or metabolizes, itraconazole.

### RELATED APPLICATIONS

This application claims the benefit of U.S. Provisional Application No. 63/402,577, filed on Aug. 31, 2022, which is incorporated herein by reference in its entirety.

### BACKGROUND OF THE INVENTION

Itraconazole is a triazole antifungal with a broad spectrum of activity, which is typically administered orally. Itraconazole is the active ingredient in the FDA-approved oral antifungal drug SPORANOX®. However, the clinical use of oral itraconazole is limited by unpredictable and variable pharmacokinetics, poor tolerability, adverse effects, and concerns related to its high drug-drug interaction (DDI) potential.

The DDI potential of itraconazole is related to its activity as a potent inhibitor of the cytochrome P450 3A4 (CYP3A4) isoenzyme. The impact of itraconazole on the CPY3A4 pathway alters the metabolism and consequently the plasma concentrations of other drugs metabolized by this pathway. Consequently, co-administration of a long list of drugs and drug classes with itraconazole is currently contraindicated due to the potential for DDIs. For example, the package insert for SPORANOX® lists over 40 contraindicated drugs and another 256 drugs for which special precautions should be taken. (See Bergagnini-Kolev, et al. The AAPS Journal (2023) 25:62; incorporated herein by reference in its entirety).

The potential DDIs of oral itraconazole greatly limit its use, particularly in patient populations that would benefit from itraconazole treatment. For example, itraconazole has shown benefits in treating allergic bronchopulmonary aspergillosis which has a prevalence of up to 15% in people with cystic fibrosis and an estimated 1.5% of patients in the general population with asthma. (See Bergagnani-Kolev, supra). However, numerous drugs used in the treatment of exacerbations of asthma or maintenance therapies for cystic fibrosis cannot be used safely with oral itraconazole (See Bergagnani-Kolev, supra).

As such, there is an unmet need for formulations and methods to treat patients with itraconazole and avoid drug-drug interactions (DDI), particularly in patients taking other medications which are substrates for CYP3A4.

### SUMMARY OF THE INVENTION

The present disclosure relates to respirable dry powders and methods for administering itraconazole to subjects for whom oral itraconazole is contraindicated, and for co-administering itraconazole with a second therapeutic agent that is contraindicated with use of oral itraconazole. In particular, the compositions and methods disclosed herein can be used to safely achieve therapeutic concentrations of itraconazole in the lungs of a patient population that is otherwise not treatable with itraconazole, such as subjects already taking a CYP3A4 substrate. For example, cystic fibrosis (CF) patients are commonly treated with medications including elexacaftor, ivacaftor, and tezacaftor, which are extensively metabolized by CYP3A4 and therefore oral itraconazole cannot be administered to those patients. This is problematic, because cystic fibrosis patients are highly susceptible to lung infections and may greatly benefit from being treated with itraconazole. The methods disclosed herein address this problem, as they may be used concomitantly with CYP3A4 substrates. In particular, the compositions and methods disclosed herein may be useful in treating allergic bronchopulmonary aspergillosis (ABPA) in subjects with cystic fibrosis (CF) or asthma, for whom oral itraconazole is contraindicated.

In some aspects, the present disclosure relates to a method of treating a disease or disorder in a subject for whom oral itraconazole is contraindicated, comprising administering to the respiratory tract of the subject a respirable dry powder comprising itraconazole. The subject can be treated with a second therapeutic agent that is a substrate, inducer, and/or inhibitor of an enzyme or receptor that is inhibited by, or metabolizes, itraconazole.

In some aspects, the present disclosure relates to a method of co-administering itraconazole with a second therapeutic agent to a subject in need thereof, wherein the itraconazole is administered as a respirable dry powder to the respiratory tract of the subject, and wherein the second therapeutic agent is a substrate, inducer, and/or inhibitor of an enzyme or receptor that is inhibited by, or metabolizes, itraconazole.

The second therapeutic agent can be a substrate, inducer, and/or inhibitor of the cytochrome P450 3A4 (CYP3A4) isoenzyme. The second therapeutic agent may be one that is contraindicated with oral itraconazole (e.g., SPORANOX®).

In some embodiments, the second therapeutic agent is an alpha blocker, a beta blocker, an analgesic, an antiarrhythmic, an antibacterial, an anticoagulant, an antiplatelet drug, an anticonvulsant, an antidiabetic drug, an anthelminthic, an antifungal, an antiprotozoal, an antimigraine drug, an antineoplastic, an antipsychotic, an anxiolytic, a hypnotic, an antiviral, a calcium channel blocker, a cardiovascular drug, a contraceptive, a diuretic, an anticonvulsant, an immunosuppressant, a lipid-lowering drug, a respiratory drug (e.g., an asthma treatment), an antidepressant drug (e.g., a tricyclic or a selective serotonin reuptake inhibitor (SSRI)), a urologic drug, a vasopressin receptor antagonist, a nonsteroidal anti-inflammatory drug (NSAID), or a gastrointestinal drug.

In some embodiments, the second therapeutic agent is alfuzosin, silodosin, tamsulosin, methadone, fentanyl, alfentanil, buprenorphine, oxycodone, sufentanil, disopyramide, dofetilide, dronedarone, quinidine, digoxin, bedaquiline, rifabutin, clarithromycin, trimetrexate, ticagrelor, apixaban, rivaroxaban, vorapaxar, cilostazol, dabigatran, warfarin, carbamazepine, repaglinidea, saxagliptin, isavuconazonium, praziquantel, artemether-lumefantrine, quinine, an ergot alkaloid (e.g., dihydroergotamine, ergometrine, ergonovine, methylergometrine, methylergonovine, ergotamine), eletriptan, irinotecan, axitinib, bosutinib, cabazitaxel, cabozantinib, ceritinib, cobimetiniba, crizotinib, dabrafenib, dasatinib, docetaxel, ibrutinib, lapatinib, nilotinib, olapariba, pazopanib, regorafenib, sunitinib, trabectedin, trastuzumab-emtansine, vinca alkaloids, bortezomib, brentuximab-vedotin, busulfan, erlotinib, gefitinib, idelalisib, nintedanib, panobinostat, ponatinib, ruxolitinib, sonidegib, vandetanib, imatinib, ixabepilone, alprazolam, aripiprazole, buspirone, diazepam, haloperidol, midazolam, quetiapine, ramelteon, risperidone, suvorexant, zopiclone, lurasidone, pimozide, triazolam, levacetylmethadol (levomethadyl), simeprevir, daclatasvir, indinavir, maraviroc, cobicistat, elvitegravir, ritonavir, saquinavir, tenofovir disoproxil fumarate, nadolol, felodipine, nisoldipine, diltiazem, dihydropyridines, verapamil, ivabradine, ranolazine, aliskiren, riociguat, sildenafil, tadalafil, bosentan, guanfacine, dienogest, ulipristal, eplerenone, cisapride, naloxegol, aprepitant, loperamide, netupitant, everolimus, sirolimus, temsirolimus, budesonide, ciclesonide, cyclosporine, dexamethasone, fluticasone, methylprednisolone, tacrolimus, lomitapide, lovastatin, simvastatin, atorvastatin, salmeterol, venlafaxine, avanafil, fesoterodine, solifenacin, darifenacin, vardenafil, dutasteride, oxybutynin, tolterodine, colchicine, eliglustat, lumacaftor, ivacaftor, elexacaftor, tezacaftor, SYMDEKO®, ORKAMBI®, KALYDECO®, alitretinoin, cabergoline, cannabinoids, cinacalcet, conivaptan, volvaptan, Saccharomyces boulardii, meloxicam, ciprofloxacin, erythromycin, clarithromycin, idelalisib, darunavir, fosamprenavir, isoniazid, rifampicin, rifabutin, phenobarbital, phenytoin, efavirenz, nevirapine, or drugs that reduce gastric acidity (e.g., acid neutralizing medicines such as aluminum hydroxide, acid secretion suppressors such as H2-receptor antagonists and proton pump inhibitors), or halofantrine.

In some embodiments, the second therapeutic agent is methadone, disopyramide, dofetilide, dronedarone, quinidine, isavuconazole, an ergot alkaloid (such as dihydroergotamine, ergometrine (ergonovine), ergotamine, methylergometrine (methylergonovine)), irinotecan, lurasidone, midazolam, pimozide, triazolam, felodipine, nisoldipine, ivabradine, ranolazine, eplerenone, cisapride, naloxegol, lomitapide, lovastatin, simvastatin, avanafil, ticagrelor, colchicine, fesoterodine, solifenacin, or eliglustat.

In a method disclosed herein, the respirable dry powder comprising itraconazole is administered to the respiratory tract of the subject at a nominal dose of between about 1 mg and about 60 mg, between about 5 mg and about 40 mg, between about 1 mg and about 10 mg, between about 10 mg and about 20 mg, between about 20 mg and about 30 mg, or between about 30 mg and about 40 mg, about 5 mg, about 10 mg, about 15 mg, about 20 mg, about 25 mg, about 30 mg, about 35 mg, or about 40 mg.

The respirable dry powder comprising itraconazole may be administered to the respiratory tract of the subject no more than about 14 days before or after administering the second therapeutic agent, less than about 14 days, less than about 12 days, less than about 10 days, less than about 8 days, less than about 7 days, less than about 6 days, less than about 5 days, less than about 4 days, less than about 3 days, less than about 2 days, or less than about 1 day before or after administering the second therapeutic agent. The respirable dry powder comprising itraconazole may be administered to the respiratory tract of the subject on the same day as administering the second therapeutic agent, less than about 20 hours, less than about 18 hours, less than about 16 hours, less than about 14 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, less than about 2 hours, less than about 1 hour, less than about 45 minutes, less than about 30 minutes, less than about 20 minutes, less than about 10 minutes, or less than about 5 minutes, before or after administering the second therapeutic agent. The respirable dry powder comprising itraconazole may be administered to the respiratory tract of the subject less than about 5 minutes before or after administering the second therapeutic agent.

In some embodiments, the respirable dry powder comprises homogenous respirable dry particles that comprise crystalline itraconazole, a stabilizer, a sodium salt, and an excipient. The sodium salt may be sodium sulfate. The stabilizer may be polysorbate 80. The excipient may be leucine.

The itraconazole can be in a crystalline sub-particle form, where the sub-particle has a size of about 50 nm to about 5,000 nm (Dv50), about 50 nm to about 800 nm (Dv50), about 50 nm to about 300 nm (Dv50), about 50 nm to about 200 nm (Dv50), or about 100 nm to about 300 nm (Dv50). The itraconazole may be present in the respirable dry particles in an amount of about 30% to about 70% by weight, about 40% to about 60% by weight, about 45%, about 50%, or about 55% by weight. In some embodiments, the itraconazole is at least 50% crystalline.

The ratio of itraconazole:stabilizer (wt:wt) in the respirable dry particles can be about 10:1.

In some embodiments, the stabilizer (e.g., polysorbate 80) is present in the respirable dry particles in an amount of about 3% to about 7% by weight. In some embodiments, the stabilizer (e.g., polysorbate 80) is present in the respirable dry particles in an amount of about 5% by weight.

In some embodiments, the excipient (e.g., leucine) is present in the respirable dry particles in an amount of about 5% to about 20% by weight. In some embodiments, the excipient (e.g., leucine) is present in the respirable dry particles in an amount of about 10% by weight.

In some embodiments, the stabilizer is polysorbate 80. In some embodiments, the excipient is leucine.

In some embodiments, the respirable dry powder comprises homogenous respirable dry particles that comprise about 50 wt % crystalline itraconazole, about 35 wt % sodium sulfate, about 10 wt % leucine, and about 5 wt % polysorbate 80.

The respirable dry particles may have: (i) a volume median geometric diameter (VMGD) of about 10 microns or less, or about 5 microns or less; (ii) a tap density of about 0.2 g/cc or greater, or a tap density of between 0.2 g/cc and 1.0 g/cc; (iii) a 1 bar/4 bar dispersibility ratio (¼ bar) of less than about 1.5, as measured by laser diffraction; and/or (iv) a 0.5 bar/4 bar dispersibility ratio (0.5/4 bar) of about 1.5 or less, as measured by laser diffraction.

The respirable dry powder may have: (i) a mass median aerodynamic diameter (MMAD) of between about 1 micron and about 5 microns; and/or (ii) a fine particle fraction (FPF) of the total dose less than 5 microns of about 25% or more.

In some embodiments, the respirable dry particles have a capsule emitted powder mass of at least 80% when emitted from a passive dry powder inhaler that has a resistance of about 0.036 sqrt (kPa)/liters per minute under the following conditions; an inhalation flow rate of 30 LPM for a period of 3 seconds using a size 3 capsule that contains a total mass of 10 mg, said total mass consisting of the respirable dry particles, and wherein the volume median geometric diameter of the respirable dry particles emitted from the inhaler as measured by laser diffraction is 5 microns or less.

In a method disclosed herein, the respirable dry powder can be delivered to the respiratory tract of the subject with a capsule-based passive dry powder inhaler.

In a method disclosed herein, the subject may have an infection, allergic bronchopulmonary aspergillosis, a respiratory disease, an acute exacerbation of a respiratory disease, an immunodeficiency disorder, cancer, a cardiovascular disorder, hypertension, hypercholesterolemia, an autoimmune disorder, diabetes, a gastrointestinal disorder, a thrombotic disorder, epilepsy, a psychiatric disorder, migraine, or pain. For example, the subject can have a fungal infection, such as aspergillosis. The subject may have cystic fibrosis, asthma, or pneumonia (e.g., fungal pneumonia). The subject may have HIV or AIDS). The subject may have a form of cancer such as lung cancer (e.g., non-small cell lung cancer). The subject may have congestive heart failure, cardiac dysrhythmias, or cardiac disease. The subject may have bipolar disorder, depression, psychosis, or anxiety. The subject may have acute pain or chronic pain. The subject may have surgical pain (e.g., perioperative pain or postoperative pain).

In some aspects, the present disclosure relates to a respirable dry powder disclosed herein for use in a method of treating a disease or disorder in a subject for whom oral itraconazole is contraindicated, wherein the respirable dry powder comprises the itraconazole and is administered to the respiratory tract of the subject. For example, the subject may be treated with a second therapeutic agent that is a substrate, inducer, and/or inhibitor of an enzyme or receptor that is inhibited by, or metabolizes, itraconazole.

In some aspects, the present disclosure relates to a respirable dry powder disclosed herein for use in a method of co-administering itraconazole and a second therapeutic agent to a subject in need thereof, wherein the respirable dry powder comprises the itraconazole and is administered to the respiratory tract of the subject, and wherein the second therapeutic agent is a substrate, inducer, and/or inhibitor of an enzyme or receptor that is inhibited by, or metabolizes, itraconazole.

In some aspects, the present disclosure relates to use of a respirable dry powder disclosed herein in the manufacture of a medicament for treating a disease or disorder in a subject for whom oral itraconazole is contraindicated, wherein the respirable dry powder comprises the itraconazole and is administered to the respiratory tract of the subject. The subject may be treated with a second therapeutic agent that is a substrate, inducer, and/or inhibitor of an enzyme or receptor that is inhibited by, or metabolizes, itraconazole.

In some aspects, the present disclosure relates to use a respirable dry powder disclosed herein in the manufacture of a medicament for co-administering itraconazole and a second therapeutic agent to a subject in need thereof, wherein the respirable dry powder comprises the itraconazole and is administered to the respiratory tract of the subject, and wherein the second therapeutic agent is a substrate, inducer, and/or inhibitor of an enzyme or receptor that is inhibited by, or metabolizes, itraconazole.

### BRIEF DESCRIPTION OF THE DRAWINGS

FIG. 1 is a diagram depicting a structural model of first-order absorption from the lungs to the systemic circulation.

FIGS. 2A and 2B are log-linear plots depicting simulated and observed plasma-concentration time profiles of itraconazole (FIG. 2A) and OH-itraconazole (FIG. 2B) after multiple doses of an exemplary itraconazole dry powder (Formulation I; 35 mg QD for 14 days) in healthy subjects. Depicted are simulated (lines) and observed data (circles; mean of n=6 individuals). The gray lines represent the 5th and 95th percentiles and the solid black line the mean data for the simulated population (n=60).

FIGS. 3A and 3B are log-linear plots depicting simulated mean plasma concentration-time profiles of itraconazole (FIG. 3A) and OH-itraconazole (FIG. 3B) after multiple oral dose of an exemplary itraconazole dry powder (Formulation I; 40 mg QD for 14 days) in healthy subjects. Depicted are simulated (lines). The gray lines represent the 5th and 95th percentiles, and the solid black line the mean data for the simulated population (n=100).

FIGS. 4A and 4B are log-linear plots depicting simulated plasma concentration-time profiles of a single 5 mg dose of midazolam, co-administered with steady state Formulation I at 35 mg (FIG. 4A) and at 40 mg (FIG. 4B) healthy subjects. Depicted are simulated midazolam plasma concentration-time profiles in the absence of Formulation I (solid line) and on the 14th day of 14 days of dosing of Formulation I (dashed line). The lines represent the mean data for the simulated population (n=100).

### DETAILED DESCRIPTION OF THE INVENTION

The present disclosure relates to methods for administering itraconazole to the respiratory tract of a subject for whom oral itraconazole is contraindicated, e.g., due to the subject being treated with a second therapeutic agent that is not usually combined with oral itraconazole due to potential DDIs. A high and consistent lung exposure with substantially lower systemic exposure can be achieved following inhaled delivery of itraconazole relative to conventional oral dosing. Without wishing to be bound by theory, it is believed that this limited systemic exposure may be beneficial for use in subjects for whom oral itraconazole is contraindicated, and using inhaled respirable dry powders containing itraconazole, it is possible to safely achieve therapeutic concentrations of itraconazole in the lungs of this patient population, e.g., to treat certain diseases or disorders affecting the respiratory system.

As such, in some aspects the present disclosure relates to a method of treating a disease or disorder in a subject for whom oral itraconazole is contraindicated, comprising administering to the respiratory tract of the subject a respirable dry powder comprising itraconazole. The subject may be treated with a second therapeutic agent that is a substrate, inducer, and/or inhibitor of an enzyme or receptor that is inhibited by, or metabolizes, itraconazole (e.g., wherein the second therapeutic agent is a substrate, inducer, and/or inhibitor of the cytochrome P450 3A4 (CYP3A4) isoenzyme). In some aspects, the present disclosure relates to a method of co-administering itraconazole and a second therapeutic agent to a subject in need thereof, wherein the itraconazole is administered as a respirable dry powder to the respiratory tract of the subject, and wherein the second therapeutic agent is a substrate, inducer, and/or inhibitor of an enzyme or receptor that is inhibited by, or metabolizes, itraconazole (e.g., wherein the second therapeutic agent is a substrate, inducer, and/or inhibitor of the cytochrome P450 3A4 (CYP3A4) isoenzyme).

### DEFINITIONS

As used herein, the term “about” refers to a relative range of plus or minus 20% of a stated value, e.g., “about 20 mg” would be “20 mg plus or minus 4 mg”.

As used herein, the terms “administration” or “administering” refer to the introduction of a therapeutic agent, or a composition comprising a therapeutic agent, to a subject. For example, administering may refer to introducing a respirable dry powder disclosed herein to the respiratory tract of a subject.

As used herein, the term “amorphous” indicates lack of significant crystallinity when analyzed via powder X-ray diffraction (XRD).

The term “capsule emitted powder mass” or “CEPM” as used herein refers to the amount of dry powder emitted from a capsule or dose unit container during actuation from the dry powder inhaler, such as during an inhalation maneuver. CEPM is measured gravimetrically, typically by weighing a capsule before and after the emission event to determine the mass of powder removed. CEPM can be expressed either as the mass of powder removed, in milligrams, or as a percentage of the initial filled powder mass in the capsule prior to the emission event.

The term “crystalline particulate form” as used herein refers to itraconazole (including pharmaceutically acceptable forms thereof including salts, polymorphs, solvates, hydrates, and the like), that is in the form of a particle (i.e., sub-particle that is smaller than the respirable dry particles that comprise the dry powders disclosed herein) and in which the itraconazole is at least about 50% crystalline. The percent crystallinity of itraconazole refers to the percentage of the compound that is in crystalline form relative to the total amount of compound present in the sub-particle. If desired, the itraconazole can be at least about 60%, at least about 70%, at least about 80%, at least about 90%, at least about 95%, or about 100% crystalline. Itraconazole in crystalline particulate form may be in the form of a particle that is about 50 nanometers (nm) to about 5,000 nm volume median diameter (Dv50), preferably 80 nm to 1750 nm Dv50, or preferably 50 nm to 800 nm Dv50.

The term “dispersible” is a term of art that describes the characteristic of a dry powder or respirable dry particles to be dispelled into a respirable aerosol. Dispersibility of a dry powder or respirable dry particles is expressed herein, in one aspect, as the quotient of the volumetric median geometric diameter (VMGD) measured at a dispersion (i.e., regulator) pressure of 1 bar divided by the VMGD measured at a dispersion (i.e., regulator) pressure of 4 bar, or VMGD at 0.5 bar divided by the VMGD at 4 bar as measured by laser diffraction, such as with a HELOS/RODOS. These quotients are referred to herein as “1 bar/4 bar dispersibility ratio” and “0.5 bar/4 bar dispersibility ratio”, respectively, and dispersibility correlates with a low quotient. For example, 1 bar/4 bar dispersibility ratio refers to the VMGD of a dry powder or respirable dry particles emitted from the orifice of a RODOS dry powder disperser (or equivalent technique) at about 1 bar, as measured by a HELOS or other laser diffraction system, divided by the VMGD of the same dry powder or respirable dry particles measured at 4 bar by HELOS/RODOS. Thus, a highly dispersible dry powder or respirable dry particles will have a 1 bar/4 bar dispersibility ratio or 0.5 bar/4 bar dispersibility ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. In another aspect, dispersibility is assessed by measuring the particle size emitted from an inhaler as a function of flowrate. As the flow rate through the inhaler decreases, the amount of energy in the airflow available to be transferred to the powder to disperse it decreases. A highly dispersible powder will have a size distribution such as is characterized aerodynamically by its mass median aerodynamic diameter (MMAD) or geometrically by its VMGD that does not substantially increase over a range of flow rates typical of inhalation by humans, such as about 15 to about 60 liters per minute (LPM), about 20 to about 60 LPM, or about 30 LPM to about 60 LPM. A highly dispersible powder will also have an emitted powder mass or dose, or a capsule emitted powder mass or dose, of about 80% or greater even at the lower inhalation flow rates. VMGD may also be called the volume median diameter (VMD), x50, or Dv50.

The term “dry particles” as used herein refers to respirable particles that may comprise up to about 15% total of water and/or another solvent. Preferably, the dry particles comprise water and/or another solvent up to about 10% total, up to about 5% total, up to about 1% total, or between 0.01% and 1% total, by weight of the dry particles, or can be substantially free of water and/or other solvent.

The term “dry powder” as used herein refers to compositions that comprise respirable dry particles. A dry powder may comprise up to about 15% total of water and/or another solvent. Preferably the dry powder comprise water and/or another solvent up to about 10% total, up to about 5% total, up to about 1% total, or between 0.01% and 1% total, by weight of the dry powder, or can be substantially free of water and/or other solvent. In one aspect, the dry powder is a respirable dry powder.

The term “effective amount,” as used herein, refers to the amount of agent needed to achieve the desired effect; such as treating a fungal infection or related disorder, e.g., allergic bronchopulmonary aspergillosis (ABPA). The actual effective amount for a particular use can vary according to the particular dry powder or respirable dry particle, the mode of administration, and the age, weight, general health of the subject, and severity of the symptoms or condition being treated. Suitable amounts of dry powders and dry particles to be administered, and dosage schedules for a particular patient can be determined by a clinician of ordinary skill based on these and other considerations.

As used herein, the term “emitted dose” or “ED” refers to an indication of the delivery of a drug formulation from a suitable inhaler device after a firing or dispersion event. More specifically, for dry powders, the ED is a measure of the percentage of powder that is drawn out of a unit dose package and that exits the mouthpiece of an inhaler device. The ED is defined as the ratio of the drug or powder delivered by an inhaler device to the nominal dose (i.e., the mass of drug or powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, MD, 13th Revision, 222-225, 2007. This method utilizes an in vitro device set up to mimic patient dosing. It can also be calculated from the results generated by Next Generation Impactor (NGI) experiments, through summation of all of the drug or powder assayed from the mouthpiece adapter, NGI induction port, and all of the stages within the NGI. The results generated through ED testing per USP 601 and the results generated via the NGI are typically in good agreement.

The term “lung to plasma ratio” or “lung: plasma ratio” refers to the ratio of a concentration of itraconazole in the lung versus the concentration of the itraconazole in the plasma at either a specific point in time or over a specific range of time. For example, the lung: plasma ratio may be calculated based on concurrent measurements at the maximum concentration (i.e., the “Cₘₐₓ”) of itraconazole in the lung or in the serum, or at any point in time. The lung: plasma ratio may also be calculated for a total exposure over a certain period of time (i.e., the “area under the curve” or “AUC”) such as over a 24 hour period. The lung concentrations of the itraconazole may be assessed by measuring the levels in the sputum, by lung lavage, by biopsy or by some other method. The lung: plasma ratio may be calculated based on concurrent measurements at any point in the dosing cycle and may be calculated based on concurrent measurements before or at steady state.

The term “nominal dose” as used herein refers to an individual dose of itraconazole. The nominal dose is the total dose of the itraconazole within one receptacle, e.g., capsule, blister, or ampule.

The terms “FPF (<X),” “FPF (<X microns),” and “fine particle fraction of less than X microns” as used herein, wherein X equals, for example, 3.4 microns, 4.4 microns, 5.0 microns or 5.6 microns, refer to the fraction of a sample of dry particles that have an aerodynamic diameter of less than X microns. For example, FPF (<X) can be determined by dividing the mass of respirable dry particles deposited on stage two and on the final collection filter of a two-stage collapsed Andersen Cascade Impactor (ACI) by the mass of respirable dry particles weighed into a capsule for delivery to the instrument. This parameter may also be identified as “FPF_TD (<X),” where TD means total dose. A similar measurement can be conducted using an eight-stage ACI. An eight-stage ACI cutoffs are different at the standard 60 L/min flowrate, but the FPF_TD (<X) can be extrapolated from the eight-stage complete data set. The eight-stage ACI result can also be calculated by the USP method of using the dose collected in the ACI instead of what was in the capsule to determine FPF. Similarly, a seven-stage Next Generation Impactor (NGI) can be used.

The terms “FPD (<X)”, “FPD<X microns”, FPD (<X microns)” and “fine particle dose of less than X microns” as used herein, wherein X equals, for example, 3.4 microns, 4.4 microns, 5.0 microns or 5.6 microns, refer to the mass of a therapeutic agent delivered by respirable dry particles that have an aerodynamic diameter of less than X micrometers. FPD<X microns can be determined by using an eight-stage Andersen Cascade Impactor (ACI) or a Next Generation Impactor (NGI) at the standard 60 L/min flowrate and summing the mass deposited on the final collection filter, and either directly calculating or extrapolating the FPD value. Similarly, a seven-stage Next Generation Impactor (NGI) can be used.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

As used herein, the term “respiratory tract” includes the upper respiratory tract (e.g., nasal passages, nasal cavity, throat, pharynx, and larynx), respiratory airways (e.g., trachea, bronchi, and bronchioles) and lungs (e.g., respiratory bronchioles, alveolar ducts, alveolar sacs, and alveoli).

The term “small” as used herein to describe respirable dry particles refers to particles that have a volume median geometric diameter (VMGD) of about 10 microns or less, preferably about 5 microns or less, or less than 5 microns.

The term “stabilizer” as used herein refers to a compound that improves the physical stability of the itraconazole in crystalline particulate form when suspended in a liquid in which the itraconazole is poorly soluble (e.g., reduces the aggregation, agglomeration, Ostwald ripening and/or flocculation of the particulates). Suitable stabilizers are surfactants and amphiphilic materials and include polysorbates (PS; polyoxyethylated sorbitan fatty acid esters), such as polysorbate 20 (PS20), polysorbate 40 (PS40), polysorbate 60 (PS60), and polysorbate 80 (PS80); fatty acids such as lauric acid, palmitic acid, myristic acid, oleic acid and stearic acid, and their salts; sorbitan fatty acid esters, such as Span20, Span40, Span60, Span80, and Span 85; phospholipids such as dipalmitoylphosphosphatidylcholine (DPPC), 1,2-dipalmitoyl-sn-glycero-3-phospho-L-serine (DPPS), 1,2-dipalmitoyl-sn-glycero-3-phosphocholine (DSPC), 1-palmitoyl-2-oleoylphosphatidylcholine (POPC), and 1,2-dioleoyl-sn-glycero-3-phosphocholine (DOPC); phosphatidylglycerols (PGs) such as diphosphatidyl glycerol (DPPG), DSPG, DPPG, POPG, etc.; 1,2-distearoyl-sn-glycero-3-phosphoethanolamine (DSPE); fatty alcohols; benzyl alcohol, polyoxyethylene-9-lauryl ether; glycocholate; surfactin; poloxomers; polyvinylpyrrolidone (PVP); PEG/PPG block co-polymers (Pluronics/Poloxamers); polyoxyethyene chloresteryl ethers; POE alky ethers; tyloxapol; lecithin; and the like. Preferred stabilizers are polysorbates and fatty acids. A particularly preferred stabilizer is polysorbate 80 (PS80).

The term “homogenous dry particles” as used herein refers to particles that are compositionally homogenous. Homogenous dry particles disclosed herein are substantially the same in their composition of itraconazole, stabilizer, and optionally one or more excipients, and exclude a blend of two or more particles.

### Methods of Administering Itraconazole Dry Powders

The inventors have discovered that administration of a dry powder comprising itraconazole disclosed herein can achieve a lung concentration of the itraconazole that is substantially greater than those achievable by oral dosing, while maintaining relatively low systemic concentration of itraconazole. Without wishing to be bound by theory, it is believed that by achieving greater lung concentration of the itraconazole using the dry powder disclosed herein, the administration can achieve therapeutic concentrations of itraconazole in the respiratory system while minimizing the systemic concentration of the itraconazole. Not only can minimizing the systemic concentration of itraconazole help to prevent side-effects and toxicity that are associated with itraconazole, but it is also possible to minimize inhibition of enzymes or receptors that itraconazole and OH-itraconazole are inhibitors or substates of, such as CYP3A4. In other words, by minimizing systemic concentration of itraconazole, the respirable dry powders of the present disclosure can be used to avoid DDIs when the subject is treated with a second therapeutic agent contraindicated with use of itraconazole, e.g., due to it being a substrate of, or an inducer or inhibitor of, the same enzymes or receptors (e.g., CYP3A4). The respirable dry powders of the present application are particularly useful for coadministration with therapeutic agents that are CYP3A4 inhibitors or inducers, and/or are metabolized by the same metabolic pathway(s) as itraconazole.

It is further believed that a therapeutic concentration of the itraconazole in the lung can be achieved even with a relatively low amount of total dose administered, e.g., relative to conventional oral administration. For example, studies have documented the systemic and pulmonary pharmacokinetics of oral itraconazole in adults and children, and pharmacokinetic studies examining respirable dry powders comprising itraconazole have shown that it is possible to achieve lung exposures that would be considered therapeutic for the treatment of pulmonary aspergillosis, after a relatively low single inhaled dose of 20 mg of dry powder. (See Conte, J. E., et al. Antimicrob. Agents Chemother. (2004) 48:3823-3827; see also Hava, D. L., et al., Brit. J. Clin. Pharmacol. (2020) 86 (4): 723-733; each of which are incorporated herein by reference in their entireties). Advantageously, the relatively low total dose of itraconazole that is needed to be administered to achieve a therapeutic effect using a dry powder disclosed herein, compared to the large amounts required for oral dosing, can reduce the risk of DDIs, which provides the opportunity to combine the dry powder comprising itraconazole with a second therapeutic agent, particularly those known to have DDIs and/or those contraindicated with itraconazole use, such as a substrate, inducer, or inhibitor of CYP3A4.

Dry powders that comprise itraconazole in amorphous form have shorter lung residence times, reduced lung to plasma exposure ratios, and undesirable toxic effects on lung tissue when inhaled at therapeutic doses. Without wishing to be bound by any particular theory, it is believed that the dry powders disclosed herein comprising crystalline forms (e.g., nanocrystalline forms) of itraconazole have a slower dissolution rate in the lung relative to the amorphous form, providing more continuous exposure over a 24 hour period after administration and minimizing systemic exposure and DDI potential.

Further, wishing to be bound by any particular theory, it is believed that smaller crystalline particles of the itraconazole (e.g., nano-crystalline or micro-crystalline itraconazole) will dissolve in the airway lining fluid more rapidly than larger crystalline particles, in part due to the larger total amount of surface area. It is also believed that crystalline itraconazole will dissolve more slowly in the airway lining fluid than the amorphous itraconazole, in part due to the lower aqueous solubility. Accordingly, the dry powders described herein can be formulated using itraconazole in crystalline particulate form that a desired crystalline size or range of crystalline sizes within the dry powders, and optionally with suitable excipients and stabilizers in a suitable ratio with the itraconazole, each of which can be tailored to affect, for example, dissolution rate, and achieve desired pharmacokinetic properties while avoiding unacceptable toxicity in the lungs in addition to avoiding DDIs with a second therapeutic agent.

Administering a dry powder disclosed herein can obtain a relatively high ratio of lung concentration:systemic concentration of the itraconazole. Without wishing to be bound by any particular theory, it is believed that a relatively high ratio of lung concentration:systemic concentration can not only minimize off-target effects and/or toxicity associated with itraconazole, but allow for coadministration with a second therapeutic agent that is contraindicated with itraconazole use and lower risk of DDIs. As such, the methods disclosed herein provide an advantage over commercially available formulations of itraconazole which are typically administered orally and in large amounts, and which cannot be co-administered with many other useful therapeutic agents due to high potential of DDIs and the associated safety concerns.

As detailed in the exemplification section herein, the impact of inhaled itraconazole on the metabolism of a second therapeutic agent that is a substrate, inducer, and/or inhibitor of an enzyme or receptor that is inhibited by, or metabolizes, itraconazole was evaluated using PBPK techniques, with midazolam as the model second therapeutic agent. Specifically, an existing oral PBPK model for itraconazole and OH-itraconazole was adapted within the Simcyp compound library, which have undergone robust verification with clinical DDI data, giving a high confidence in the model predictions for respirable dry powders comprising itraconazole. Despite pharmacologic concentrations of itraconazole in the tissues of the lungs, there is minimal itraconazole exposure in the intestines and liver. As a result, there was minimal impact predicted on the metabolism of midazolam, despite the overprediction of the primary active metabolite, OH-itraconazole. These results indicate that itraconazole can be safely and effectively administered via an inhaled formulation even in patients taking a second therapeutic agent that is substrate, inducer, and/or inhibitor of an enzyme or receptor that is inhibited by, or metabolizes, itraconazole (e.g., CYP3A4). The clinical implications of being able to safely administer itraconazole with one of those second therapeutic agents are clear, and should improve the treatment possibilities for vulnerable patients maintained on medications that are contraindicated in the presence of oral itraconazole.

Methods disclosed herein may comprise treating a disease or disorder in a subject for whom oral itraconazole is contraindicated, comprising administering to the respiratory tract of the subject a respirable dry powder comprising itraconazole (e.g., crystalline itraconazole). Additionally, methods disclosed herein may comprise co-administering itraconazole with a second therapeutic agent to a subject in need thereof, wherein the itraconazole is administered as a respirable dry powder to the respiratory tract of the subject.

The second therapeutic agent may be a substrate of an enzyme or receptor that is inhibited by, or metabolizes, itraconazole (e.g., CYP3A4). The second therapeutic agent may be an inhibitor of an enzyme or receptor that is inhibited by, or metabolizes, itraconazole (e.g., CYP3A4). The second therapeutic agent may be an inducer of an enzyme or receptor that is inhibited by, or metabolizes, itraconazole (e.g., CYP3A4).

The second therapeutic agent may be contraindicated with oral itraconazole (e.g., SPORANOX®). For example, the second therapeutic agent may be a substance that is listed as contraindicated for use with itraconazole by a regulatory authority, e.g., as stated in the FDA label for SPORANOX®.

There are many different classes of drugs that are contraindicated, or otherwise not recommended to be combined with oral itraconazole at normal doses. As such, the respirable dry powders disclosed herein may be combined with many different classes of drugs, without significant risk of DDI or other adverse reactions, and/or without requiring careful adjustment or limiting doses to avoid potential DDIs or adverse reactions.

For example, a method disclosed herein may comprise coadministration of the respirable dry powder with, or administration of the respirable dry powder to a subject that is also administered with, a second therapeutic agent selected from the group consisting of an alpha blocker, a beta blocker, an analgesic, an antiarrhythmic, an antibacterial, an anticoagulant, an antiplatelet drug, an anticonvulsant, an antidiabetic drug, an anthelminthic, an antifungal, an antiprotozoal, an antimigraine drug, an antineoplastic, an antipsychotic, an anxiolytic, a hypnotic, an antiviral, a calcium channel blocker, a cardiovascular drug, a contraceptive, a diuretic, an anticonvulsant, an immunosuppressant, a lipid-lowering drug, a respiratory drug (e.g., an asthma treatment), an antidepressant drug (e.g., a tricyclic or a selective serotonin reuptake inhibitor (SSRI)), a urologic drug, a vasopressin receptor antagonist, a nonsteroidal anti-inflammatory drug (NSAID), or a gastrointestinal drug.

The second therapeutic agent may be alfuzosin, silodosin, or tamsulosin. The second therapeutic agent may be methadone, fentanyl, alfentanil, buprenorphine, oxycodone, or sufentanil. The second therapeutic agent may be disopyramide, dofetilide, dronedarone, quinidine, or digoxin. The second therapeutic agent may be bedaquiline, rifabutin, clarithromycin, or trimetrexate. The second therapeutic agent may be ticagrelor, apixaban, rivaroxaban, vorapaxar, cilostazol, dabigatran, warfarin, or carbamazepine. The second therapeutic agent may be repaglinidea or saxagliptin. The second therapeutic agent may be isavuconazonium, praziquantel, artemether-lumefantrine, or quinine. The second therapeutic agent may be an ergot alkaloid, such as dihydroergotamine, ergometrine, ergonovine, methylergometrine, methylergonovine, ergotamine. The second therapeutic agent may be eletriptan. The second therapeutic agent may be irinotecan, axitinib, bosutinib, cabazitaxel, cabozantinib, ceritinib, cobimetiniba, crizotinib, dabrafenib, dasatinib, docetaxel, ibrutinib, lapatinib, nilotinib, olapariba, pazopanib, regorafenib, sunitinib, trabectedin, trastuzumab-emtansine, vinca alkaloids, bortezomib, brentuximab-vedotin, busulfan, erlotinib, gefitinib, idelalisib, nintedanib, panobinostat, ponatinib, ruxolitinib, sonidegib, vandetanib, imatinib, or ixabepilone. The second therapeutic agent may be alprazolam, aripiprazole, buspirone, diazepam, haloperidol, midazolam, quetiapine, ramelteon, risperidone, suvorexant, zopiclone, lurasidone, pimozide, or triazolam. The second therapeutic agent may be levacetylmethadol (levomethadyl), simeprevir, daclatasvir, indinavir, maraviroc, cobicistat, elvitegravir, ritonavir, saquinavir, or tenofovir disoproxil fumarate. The second therapeutic agent may be nadolol. The second therapeutic agent may be felodipine, nisoldipine, diltiazem, dihydropyridines, or verapamil. The second therapeutic agent may be ivabradine, ranolazine, aliskiren, riociguat, sildenafil, tadalafil, bosentan, guanfacine, dienogest, or ulipristal. The second therapeutic agent may be eplerenone. The second therapeutic agent may be cisapride, naloxegol, aprepitant, loperamide, or netupitant. The second therapeutic agent may be everolimus, sirolimus, temsirolimus, budesonide, ciclesonide, cyclosporine, dexamethasone, fluticasone, methylprednisolone, or tacrolimus. The second therapeutic agent may be lomitapide, lovastatin, simvastatin, or atorvastatin. The second therapeutic agent may be salmeterol. The second therapeutic agent may be venlafaxine. The second therapeutic agent may be avanafil, fesoterodine, solifenacin, darifenacin, vardenafil, dutasteride, oxybutynin, or tolterodine. The second therapeutic agent may be colchicine, eliglustat, lumacaftor, ivacaftor, elexacaftor, tezacaftor, SYMDEKO®, ORKAMBI®, KALYDECO®, alitretinoin, or cabergoline. The second therapeutic agent may be a cannabinoid. The second therapeutic agent may be cinacalcet, conivaptan, or volvaptan. The second therapeutic agent may be a Saccharomyces boulardii composition. The second therapeutic agent may be meloxicam. The second therapeutic agent may be ciprofloxacin, erythromycin, or clarithromycin. The second therapeutic agent may be idelalisib. The second therapeutic agent may be darunavir or fosamprenavir. The second therapeutic agent may be isoniazid, rifampicin, or rifabutin. The second therapeutic agent may be phenobarbital, phenytoin, efavirenz, or nevirapine. The second therapeutic agent may be a drug that reduces gastric acidity, such as an acid neutralizing medicines, e.g., aluminum hydroxide, an acid secretion suppressors such as H2-receptor antagonists, or a proton pump inhibitor. The second therapeutic agent may be halofantrine.

In some embodiments, the second therapeutic agent is methadone, disopyramide, dofetilide, dronedarone, quinidine, isavuconazole, an ergot alkaloid (such as dihydroergotamine, ergometrine (ergonovine), ergotamine, or methylergometrine (methylergonovine)), irinotecan, lurasidone, midazolam, pimozide, triazolam, felodipine, nisoldipine, ivabradine, ranolazine, eplerenone, cisapride, naloxegol, lomitapide, lovastatin, simvastatin, avanafil, ticagrelor, colchicine, fesoterodine, solifenacin, or eliglustat.

In a method disclosed herein, the respirable dry powder may be administered at a nominal dose of between about 1 mg and about 60 mg, e.g., between about 5 mg and about 40 mg, between about 1 mg and about 10 mg, between about 10 mg and about 20 mg, between about 20 mg and about 30 mg, or between about 30 mg and about 40 mg, about 5 mg, about 10 mg, about 15 mg, about 20 mg, about 25 mg, about 30 mg, about 35 mg, or about 40 mg.

Coadministration may refer to administering the respirable dry powder to the subject no more than about 14 days before or after administering the second therapeutic agent, e.g., less than about 14 days, less than about 12 days, less than about 10 days, less than about 8 days, less than about 7 days, less than about 6 days, less than about 5 days, less than about 4 days, less than about 3 days, less than about 2 days, or less than about 1 day before or after administering the second therapeutic agent. In some embodiments, coadministration refers to administering the dry powder to the subject on the same day as administering the second therapeutic agent, e.g., administering the respirable dry powder less than about 20 hours, less than about 18 hours, less than about 16 hours, less than about 14 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, less than about 2 hours, less than about 1 hour, less than about 45 minutes, less than about 30 minutes, less than about 20 minutes, less than about 10 minutes, or less than about 5 minutes, before or after administering the second therapeutic agent. In some embodiments, the itraconazole is administered to the subject less than about 5 minutes before or after administering the second therapeutic agent.

In a method disclosed herein, the subject to be treated with the itraconazole may be a subject for whom oral itraconazole is contraindicated. That may be due to the subject having been administered the second therapeutic agent disclosed herein (e.g., a therapeutic agent that is contraindicated with itraconazole use). Due to the favorable pharmacokinetic properties achieved using the respirable dry powder disclosed herein, the subject may nevertheless be administered a respirable dry powder disclosed herein to achieve therapeutic local concentrations of itraconazole in the lungs, in a safe manner and avoiding DDIs or adverse events that would be expected when a different formulation of itraconazole, e.g., oral itraconazole, is administered.

The subject to be treated with the itraconazole, who may be a subject for whom oral itraconazole is contraindicated, may have a disease or disorder. The particular disease or disorder may be a condition that the itraconazole is being administered to treat, or it may be a condition that is not being treated with itraconazole, or a condition that is unrelated to the use of the itraconazole. The subject may have an infection (e.g., a fungal infection, such as aspergillosis), allergic bronchopulmonary aspergillosis, a respiratory disease (e.g., cystic fibrosis, asthma, pneumonia (e.g., fungal pneumonia)), an acute exacerbation of a respiratory disease, an immunodeficiency disorder (e.g., HIV or AIDS), cancer (e.g., lung cancer, such as non-small cell lung cancer), a cardiovascular disorder (e.g., congestive heart failure, cardiac dysrhythmias, cardiac disease), hypertension, hypercholesterolemia, an autoimmune disorder, diabetes, a gastrointestinal disorder, a thrombotic disorder, epilepsy, a psychiatric disorder (e.g., bipolar disorder, depression, psychosis, anxiety), migraine, pain (e.g., acute pain, pain caused by surgery, or chronic pain). In some cases, the disease or disorder is a condition that the itraconazole is used to treat, such as an infection (e.g., a fungal infection, such as aspergillosis), allergic bronchopulmonary aspergillosis, a respiratory disease (e.g., cystic fibrosis, asthma, pneumonia (e.g., fungal pneumonia)), an acute exacerbation of a respiratory disease, or cancer (e.g., lung cancer, such as non-small cell lung cancer).

In some embodiments, a method disclosed herein is for treating an infection (e.g., a fungal infection, such as aspergillosis), allergic bronchopulmonary aspergillosis, a respiratory disease (e.g., cystic fibrosis, asthma, pneumonia (e.g., fungal pneumonia)), an acute exacerbation of a respiratory disease, or cancer (e.g., lung cancer, such as non-small cell lung cancer) in a subject in need thereof.

### Respirable Dry Powders

The dry powders disclosed herein may be administered to a subject by inhalation, such as oral inhalation. To achieve oral inhalation, a dry powder inhaler may be used, such as a passive dry powder inhaler. Respirable dry powders comprising itraconazole for use in treating a fungal infection have been described in WO 2018/071757, WO 2019/204583, and WO 2019/204597, the entire contents of which are incorporated herein by reference in their entireties.

The respirable dry powders used in the methods disclosed herein may include homogenous respirable dry particles that comprise 1) itraconazole in crystalline particulate form, 2) a stabilizer, and optionally 3) one or more excipients. Such respirable dry particles can be prepared using any suitable method, such as by preparing a feedstock in which itraconazole in crystalline particulate form is suspended in an aqueous solution of excipients, and spray drying the feedstock.

The respirable dry particles may comprise itraconazole in an amount of about 1% to about 95% by weight (wt %). It is preferred that the respirable dry particle comprises an amount of itraconazole so that a therapeutically effective dose can be administered and maintained without the need to inhale large volumes of dry powder, and also without the need to inhale the dry powder too frequently, e.g., more than three time a day. For example, it is preferred that the respirable dry particles comprise about 30% to about 70%, or about 40% to about 60%, e.g., about 45%, about 50%, or about 55% itraconazole by weight (wt %). The amount of itraconazole present in the respirable dry particles by weight may also be referred to as the “drug load.”

The itraconazole may be present in the respirable dry particles in crystalline particulate form (e.g., nano-crystalline). More specifically, in the form of a sub-particle that is about 50 nm to about 5,000 nm (Dv50), preferably, with the itraconazole being at least 50% crystalline. For example, for any desired load of the itraconazole (sometimes referred to as “drug load”), the sub-particle size can be about 100 nm, about 300 nm, about 1500 nm, about 80 nm to about 300 nm, about 80 nm to about 250 nm, about 80 nm to about 200 nm, about 100 nm to about 150 nm, about 1200 nm to about 1500 nm, about 1500 nm to about 1750 nm, about 1200 nm to about 1400 nm, or about 1200 nm to about 1350 nm (Dv50). In particular embodiments, the sub-particle is between about 50 nm to about 2500 nm, between about 80 and 1750 nm, between about 50 nm and 1000 nm, between about 50 nm and 800 nm, between about 50 nm and 600 nm, between about 50 nm and 500 nm, between about 50 nm and 400 nm, between about 50 nm and 300 nm, between about 50 nm and 200 nm, or between about 100 nm and 300 nm. In addition, for any desired drug load and sub-particle size, the degree of itraconazole crystallinity can be at least about 50%, at least about 60%, at least about 70%, at least about 80%, at least about 90%, at least about 95%, or about 100% crystalline. Preferably, the itraconazole is about 100% crystalline. In some embodiments, the dry powder administered comprises homogenous respirable dry particles that comprise itraconazole that is at least 50% crystalline, e.g., 55% crystalline, 60% crystalline, 65% crystalline, 70% crystalline, 75% crystalline, 80% crystalline, 85% crystalline, 90% crystalline, 95% crystalline, 96% crystalline, 97% crystalline, 98% crystalline, 99% crystalline, or more than 99% crystalline

The itraconazole in crystalline particulate form can be prepared in any desired sub-particle size using a suitable method, including a stabilizer if desired, such as by wet milling, jet milling or other suitable method.

The respirable dry particles also include a stabilizer. The stabilizer helps maintain the desired size of the itraconazole in crystalline particulate form during wet milling, in spray drying feedstock, and aids in wetting and dispersing and maintaining the physical stability of the itraconazole crystalline particulate suspension. It is preferred to use as little stabilizer as is needed to achieve the aforementioned benefits. The amount of stabilizer is typically in a fixed ratio to the amount of itraconazole present in the dry particle and can range from about 1:1 (itraconazole: stabilizer (wt:wt)) to about 50:1 (wt:wt), about 10:1 being preferred. For example, the ratio of itraconazole:stabilizer (wt:wt) in the dry particles can be about 8:1, about 9:1, about 10:1, about 11:1, or about 12:1.

The amount of stabilizer that is present in the dry particles can be in a range of about 1 wt % to about 15 wt %, such as about 3 wt % to about 7 wt %, or about 5 wt %. It is generally preferred that the respirable dry particles comprise less than about 10% stabilizer by weight (wt %), such as 9 wt % or less, 8 wt % or less, 7 wt % or less, 5 wt % or less, 4 wt % or less, 3 wt % or less, 2 wt % or less, or 1 wt % or less. A particularly preferred stabilizer for use in the dry powders described herein is polysorbate 80. In contrast to conventional dry powders which use surfactant to prevent the onset of crystallization in the dry powder, the surfactant in the presently disclosed dry powders is added to stabilize a colloidal suspension of the crystalline itraconazole in an anti-solvent.

In some embodiments, the dry powder administered comprises homogenous respirable dry particles that comprise itraconazole and polysorbate 80, wherein the ratio of itraconazole:polysorbate 80 (wt:wt) is about 10:1.

The respirable dry particles also include a sodium salt (e.g., sodium sulfate or sodium chloride). For example, the dry particles may comprise sodium sulfate. In preferred embodiments, the respirable dry particles comprise about 15 wt % to about 50 wt % of a sodium salt (e.g., sodium sulfate). For example, the respirable dry particles can comprise between about 25 wt % and about 45 wt % sodium salt, e.g., about 30 wt %, 35 wt %, or 40 wt % sodium salt (e.g., sodium sulfate).

The respirable dry particles also include any suitable and desired amount of one or more excipients. In some embodiments, the one or more excipients are present in an amount of about 5% to about 20% by weight. Many excipients are well-known in the art and can be included in the dry powders and dry particles described herein. Pharmaceutically acceptable excipients that are particularly preferred for the dry powders and dry particles described herein include leucine. For example, the respirable dry particles comprise an excipient (e.g., leucine), in an amount of about 1 wt % to about 20 wt %, e.g., between about 5 wt % and about 20 wt %, e.g., about 10 wt %. In some embodiments, the respirable dry particles comprise leucine in an amount of about 10 wt %.

Without wishing to be bound by theory, it is believed that combining the itraconazole in a dry powder with leucine and a sodium salt (e.g., sodium sulfate) can provide optimal dissolution rates for obtaining effective therapeutic levels of the itraconazole in the lungs without unacceptable toxicity or DDIs when the dry powder is coadministered with a second therapeutic agent. Additionally, maintaining a relatively high drug load (e.g., about 50 wt %) of the itraconazole may prevent rapid dissolution of the dry powder in the lungs. For example, the dry powders disclosed herein may dissolve in the lungs more slowly, compared to a formulation combining relatively low amounts of itraconazole (e.g., less than 40 wt %) with a hydrophilic excipient such as mannitol.

The dissolution of dry powders used in the methods disclosed herein may be measured in terms of the dissolution half-life. In some embodiments, the dry powders used in a method disclosed herein have a dissolution half-life that is at least about 2 minutes, e.g., between about 2 minutes and about 20 minutes, e.g., about 2 minutes, about 3 minutes, about 4 minutes, about 5 minutes, about 6 minutes, about 7 minutes, about 8 minutes, about 9 minutes, or about 10 minutes. In some embodiments, the dissolution half-life is about 4.1 minutes, about 4.2 minutes, about 4.3 minutes, or about 4.4 minutes. In some embodiments the dissolution half-life is between about 4.13 minutes and about 16.84 minutes.

In one aspect, the dry powder comprises respirable dry particles comprising: (i) about 50 wt % itraconazole in crystalline particulate form, about 5 wt % of a stabilizer, about 35 wt % of a sodium salt, and about 10 wt % leucine. For example, the dry powder may comprise respirable dry particles comprising: (i) 50 wt % itraconazole in crystalline particulate form, 5 wt % of a stabilizer (e.g., polysorbate 80), 35 wt % of a sodium salt (e.g., sodium sulfate), and 10 wt % leucine. The dry powder may consist essentially of respirable dry particles that consist essentially of: (i) 50 wt % itraconazole in crystalline particulate form, 5 wt % of a stabilizer (e.g., polysorbate 80), 35 wt % of a sodium salt (e.g., sodium sulfate), and 10 wt % leucine.

The dry powders disclosed herein may be free of lactose or other carrier particles.

The dry powders and/or respirable dry particles are preferably small, mass dense, and dispersible. To measure volumetric median geometric diameter (VMGD), a laser diffraction system may be used, e.g., a Spraytec system (particle size analysis instrument, Malvern Instruments) and a HELOS/RODOS system (laser diffraction sensor with dry dispensing unit, Sympatec GmbH). The respirable dry particles have a VMGD as measured by laser diffraction at the dispersion pressure setting (also called regulator pressure) of 1.0 bar at a maximum orifice ring pressure using a HELOS/RODOS system of about 10 microns or less, about 5 microns or less, about 4 μm or less, about 3 μm or less, about 1 μm to about 5 μm, about 1 μm to about 4 μm, about 1.5 μm to about 3.5 μm, about 2 μm to about 5 μm, about 2 μm to about 4 μm, or about 2 μm to about 3 μm. Preferably, the VMGD is about 5 microns or less, or about 4 μm or less. In one aspect, the dry powders and/or respirable dry particles have a minimum VMGD of about 0.5 microns or about 1.0 micron.

The dry powders and/or respirable dry particles preferably have 1 bar/4 bar dispersibility ratio and/or 0.5 bar/4 bar dispersibility ratio of less than about 2.0 (e.g., about 0.9 to less than about 2), about 1.7 or less (e.g., about 0.9 to about 1.7) about 1.5 or less (e.g., about 0.9 to about 1.5), about 1.4 or less (e.g., about 0.9 to about 1.4), or about 1.3 or less (e.g., about 0.9 to about 1.3), and preferably have a 1 bar/4 bar and/or a 0.5 bar/4 bar of about 1.5 or less (e.g., about 1.0 to about 1.5), and/or about 1.4 or less (e.g., about 1.0 to about 1.4).

The dry powders and/or respirable dry particles preferably have a tap density of at least about 0.2 g/cm³, of at least about 0.25 g/cm³, a tap density of at least about 0.3 g/cm³, of at least about 0.35 g/cm³, a tap density of at least 0.4 g/cm³. For example, the dry powders and/or respirable dry particles have a tap density of greater than 0.4 g/cm³ (e.g., greater than 0.4 g/cm³ to about 1.2 g/cm³), a tap density of at least about 0.45 g/cm³ (e.g., about 0.45 g/cm³ to about 1.2 g/cm³), at least about 0.5 g/cm³ (e.g., about 0.5 g/cm³ to about 1.2 g/cm³), at least about 0.55 g/cm³ (e.g., about 0.55 g/cm³ to about 1.2 g/cm³), at least about 0.6 g/cm³ (e.g., about 0.6 g/cm³ to about 1.2 g/cm³) or at least about 0.6 g/cm³ to about 1.0 g/cm³. Alternatively, the dry powders and/or respirable dry particles preferably have a tap density of about 0.01 g/cm³ to about 0.5 g/cm³, about 0.05 g/cm³ to about 0.5 g/cm³, about 0.1 g/cm³ to about 0.5 g/cm³, about 0.1 g/cm³ to about 0.4 g/cm³, or about 0.1 g/cm³ to about 0.4 g/cm³. Alternatively, the dry powders and/or respirable dry particles have a tap density of about 0.15 g/cm³ to about 1.0 g/cm³. Alternatively, the dry powders and/or respirable dry particles have a tap density of about 0.2 g/cm³ to about 0.8 g/cm³.

The dry powders and/or respirable dry particles have a bulk density of at least about 0.1 g/cm³, or at least about 0.8 g/cm³. For example, the dry powders and/or respirable dry particles have a bulk density of about 0.1 g/cm³ to about 0.6 g/cm³, about 0.2 g/cm³ to about 0.7 g/cm³, about 0.3 g/cm³ to about 0.8 g/cm³.

The respirable dry particles, and the dry powders when the dry powders are respirable dry powders, preferably have an MMAD of less than 10 microns, preferably an MMAD of about 5 microns or less, or about 4 microns or less. In one aspect, the respirable dry powders and/or respirable dry particles preferably have a minimum MMAD of about 0.5 microns, or about 1.0 micron. In one aspect, the respirable dry powders and/or respirable dry particles preferably have a minimum MMAD of about 2.0 microns, about 3.0 microns, or about 4.0 microns.

The dry powders and/or respirable dry particles preferably have a FPF of less than about 5.6 microns (FPF<5.6 μm) of the total dose of at least about 35%, preferably at least about 45%, at least about 60%, between about 45% to about 80%, or between about 60% and about 80%.

The dry powders and/or respirable dry particles preferably have a FPF of less than about 3.4 microns (FPF<3.4 μm) of the total dose of at least about 20%, preferably at least about 25%, at least about 30%, at least about 40%, between about 25% and about 60%, or between about 40% and about 60%.

The dry powders and/or respirable dry particles preferably have a total water and/or solvent content of up to about 15% by weight, up to about 10% by weight, up to about 5% by weight, up to about 1%, or between about 0.01% and about 1%, or may be substantially free of water or other solvent.

The dry powders and/or respirable dry particles preferably may be administered with low inhalation energy. In order to relate the dispersion of powder at different inhalation flow rates, volumes, and from inhalers of different resistances, the energy required to perform the inhalation maneuver may be calculated. Inhalation energy can be calculated from the equation E=R²Q²V where E is the inhalation energy in Joules, R is the inhaler resistance in kPa^{1/2}/LPM, Q is the steady flow rate in L/min and V is the inhaled air volume in L.

Healthy adult populations are predicted to be able to achieve inhalation energies ranging from 2.9 Joules for comfortable inhalations to 22 Joules for maximum inhalations by using values of peak inspiratory flow rate (PIFR) measured by Clarke et al. (Journal of Aerosol Med, 6(2), p.99-110, 1993) for the flow rate Q from two inhaler resistances of 0.02 and 0.055 kPa^{1/2}/LPM, with an inhalation volume of 2 L based on both FDA guidance documents for dry powder inhalers and on the work of Tiddens et al. (Journal of Aerosol Med, 19(4), p.456-465, 2006) who found adults averaging 2.2 L inhaled volume through a variety of DPIs.

Mild, moderate and severe adult COPD patients are predicted to be able to achieve maximum inhalation energies of 5.1 to 21 Joules, 5.2 to 19 Joules, and 2.3 to 18 Joules respectively. This is again based on using measured PIFR values for the flow rate Q in the equation for inhalation energy. The PIFR achievable for each group is a function of the inhaler resistance that is being inhaled through. The work of Broeders et al. (Eur Respir J, 18, p.780-783, 2001) was used to predict maximum and minimum achievable PIFR through two dry powder inhalers of resistances 0.021 and 0.032 kPa^{1/2}/LPM for each.

Similarly, adult asthmatic patients are predicted to be able to achieve maximum inhalation energies of 7.4 to 21 Joules based on the same assumptions as the COPD population and PIFR data from Broeders et al.

Healthy adults and children, for example, are capable of providing sufficient inhalation energy to disperse a dry powder of the present disclosure, e.g., from a suitable inhalation device (e.g., dry powder inhaler).

The dry powders and/or respirable dry particles useful in a method disclosed herein are preferably characterized by a high emitted dose, such as a CEPM of at least 75%, at least 80%, at least 85%, at least 90%, at least 95%, from a passive dry powder inhaler subject to a total inhalation energy of about 5 Joules, about 3.5 Joules, about 2.4 Joules, about 2 Joules, about 1 Joule, about 0.8 Joules, about 0.5 Joules, or about 0.3 Joules is applied to the dry powder inhaler. The receptacle holding the dry powders and/or respirable dry particles may comprise about 5 mg, about 7.5 mg, about 10 mg, about 15 mg, about 20 mg, or about 30 mg. In one aspect, the dry powders and/or respirable dry particles are characterized by a CEPM of 80% or greater and a VMGD of 5 microns or less when emitted from a passive dry powder inhaler having a resistance of about 0.036 sqrt (kPa)/liters per minute under the following conditions: an air flow rate of 30 LPM, run for 3 seconds using a size 3 capsule that comprises a total mass of 10 mg. In another aspect, the dry powders and/or respirable dry particles are characterized by a CEPM of 80% or greater and a VMGD of 5 microns or less when emitted from a passive dry powder inhaler having a resistance of about 0.036 sqrt (kPa)/liters per minute under the following conditions: an air flow rate of 20 LPM, run for 3 seconds using a size 3 capsule that comprises a total mass of 10 mg. In a further aspect, the dry powders and/or respirable dry particles are characterized by a CEPM of 80% or greater and a VMGD of 5 microns or less when emitted from a passive dry powder inhaler having a resistance of about 0.036 sqrt (kPa)/liters per minute under the following conditions: an air flow rate of 15 LPM, run for 4 seconds using a size 3 capsule that comprises a total mass of 10 mg.

The dry powder can fill the unit dose container, or the unit dose container can be at least 2% full, at least 5% full, at least 10% full, at least 20% full, at least 30% full, at least 40% full, at least 50% full, at least 60% full, at least 70% full, at least 80% full, or at least 90% full. The unit dose container can be a capsule (e.g., size 000, 00, 0E, 0, 1, 2, 3, and 4, with respective volumetric capacities of 1.37 mL, 950 μL, 770 μL, 680 μL, 480 μL, 360 μL, 270 μL, and 200 μL). The capsule can be at least about 2% full, at least about 5% full, at least about 10% full, at least about 20% full, at least about 30% full, at least about 40% full, or at least about 50% full. The unit dose container can be a blister. The blister can be packaged as a single blister or as part of a set of blisters, for example, 7 blisters, 14 blisters, 28 blisters or 30 blisters. The one or more blister can be preferably at least 30% full, at least 50% full or at least 70% full.

An advantage of the dry powders disclosed herein is that they disperse well across a wide range of flow rates and are relatively flowrate independent. The dry powders and/or respirable dry particles permit the use of a simple, passive DPI for a wide patient population.

The dry powders and/or respirable dry particles useful in a method described herein are preferably characterized by: 1) a VMGD at 1 bar as measured using a HELOS/RODOS system of about 10 microns or less, preferably about 5 microns or less; 2) a 1 bar/4 bar dispersibility ratio and/or a 0.5 bar/4 bar dispersibility ratio of about 1.5 or less, about 1.4 or less or about 1.3 or less; 3) a MMAD of about 10 microns or less, preferably about 5 microns or less; 4) a FPF<5.6 μm of the total dose of at least about 45% or at least about 60%; and/or 5) a FPF<3.4 μm of the total dose of at least about 25% or at least about 40%. If desired, the dry powders and/or respirable dry particles are further characterized by a tap density of about 0.2 g/cm³ or greater, about 0.3 g/cm³ or greater, about 0.4 g/cm³ or greater, greater than 0.4 g/cm³, about 0.45 g/cm³ or greater or about 0.5 g/cm³ or greater.

Formulation I is an exemplary dry powder that can be used in a method disclosed herein. The composition and properties of Formulation I are provided below in Table 1.

TABLE 1
Composition and properties of Formulation I.

| Amount of | | | | | |
|---|---|---|---|---|---|
| crystalline | | | | Intraconazole | Intraconazole |
| itraconazole | | | | subparticle size | subparticle size |
| (crystalline) | Sodium salt | Excipient | Stabilizer | (Dv50) | range (Dv50) |
| 50 wt % | Sodium sulfate | Leucine | Polysorbate | 132 nm | 60-175 nm |
| | (35 wt %) | (10 wt %) | 80 (5 wt %) | | |

Formulation I has a fine particle fraction (FPF) less than 5 microns of the total dose of 57%, leading to a fine particle dose less than 5 microns of 2.8 mg for a 10.0 mg total dry powder capsule fill.

Formulation I has a dissolution half-life of 4.35 mins, as determined by the following protocol: powder formulations, capsules and packaging materials were equilibrated at 22.5+2.5 ° C and 30±5% RH. Formulation I was encapsulated into a size 3 HPMC capsule under the same conditions. The fill weight for the powder preparation was 10 mg. The formulation were aerosolized from the capsule in a unit-dose, capsule-based DPI device (RS01, Plastiape, Osnago, Italy), at 60 L/min (4 L inhaled volume) using the Plastiape RS01 dry powder inhaler (DPI). The aerosol dose was collected in the UniDose system. The UniDose collection system was used to uniformly deposit the whole impactor stage mass (i.e., below stage 2 of an NGI) onto a glass microfiber filter membrane, which can be seen as where the circles (representing particles or droplets) deposit. The filter was placed into a disk cassette and dissolution studies were undertaken using 500 ml PBS pH 7.4+2.0% SDS in a USP Apparatus II POD (Paddle Over Disk, USP V) at 37 ° C Sink conditions were maintained within the vessel. Samples were taken at specified time points and tested for drug content on an Agilent (Santa Clara, CA, USA) 1260 Infinity series HPLC.

The dry powders and/or respirable dry particles disclosed herein may be filled into a receptacle, for example a capsule or a blister. When the receptacle is a capsule, the capsule is, for example, a size 2 or a size 3 capsule, and is preferably a size 3 capsule. The capsule material may be, for example, gelatin or HPMC (hydroxypropyl methylcellulose), and is preferably HPMC.

The dry powder and/or respirable dry particles described and characterized herein be contained in a dry powder inhaler (DPI). The DPI may be a capsule-based DPI or a blister-based DPI, and is preferably a capsule-based DPI. More preferably, the dry powder inhaler is selected from the RS01™ family of dry powder inhalers (Plastiape S.p.A., Italy). More preferably, the dry powder inhaler is selected from the RS01™ HR or the RS01™ UHR2. Most preferably, the dry powder inhaler is the RS01™ HR.

### Methods for Preparing Dry Powders and Dry Particles

The respirable dry particles and dry powders for use in a method disclosed herein can be prepared using any suitable method, with the proviso that the dry powders cannot be an extemporaneous dispersion. Many suitable methods for preparing dry powders and/or respirable dry particles are conventional in the art, and include single and double emulsion solvent evaporation, spray drying, spray-freeze drying, milling (e.g., jet milling), blending, solvent extraction, solvent evaporation, phase separation, simple and complex coacervation, interfacial polymerization, suitable methods that involve the use of supercritical carbon dioxide (CO₂), sonocrystallization, nanoparticle aggregate formation and other suitable methods, including combinations thereof. Respirable dry particles can be made using methods for making microspheres or microcapsules known in the art. These methods can be employed under conditions that result in the formation of respirable dry particles with desired aerodynamic properties (e.g., aerodynamic diameter and geometric diameter). If desired, respirable dry particles with desired properties, such as size and density, can be selected using suitable methods, such as sieving.

Suitable methods for selecting respirable dry particles with desired properties, such as size and density, include wet sieving, dry sieving, and aerodynamic classifiers (such as cyclones).

The respirable dry particles are preferably spray dried. Suitable spray-drying techniques are described, for example, by K. Masters in “Spray Drying Handbook”, John Wiley & Sons, New York (1984). Generally, during spray-drying, heat from a hot gas such as heated air or nitrogen is used to evaporate a solvent from droplets formed by atomizing a continuous liquid feed. When hot air is used, the moisture in the air is at least partially removed before its use. When nitrogen is used, the nitrogen gas can be run “dry”, meaning that no additional water vapor is combined with the gas. If desired the moisture level of the nitrogen or air can be set before the beginning of spray dry run at a fixed value above “dry” nitrogen. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the dry particles to be produced in a suitable solvent (e.g., aqueous solvent, organic solvent, aqueous-organic mixture or emulsion) are distributed to a drying vessel via an atomization device. For example, a nozzle or a rotary atomizer may be used to distribute the solution or suspension to the drying vessel. The nozzle can be a two-fluid nozzle, which can be in an internal mixing setup or an external mixing setup. Alternatively, a rotary atomizer having a 4- or 24-vaned wheel may be used. Examples of suitable spray dryers that can be outfitted with a rotary atomizer and/or a nozzle, include, a Mobile Minor Spray Dryer or the Model PSD-1, both manufactured by GEA Niro, Inc. (Denmark), Büchi B-290 Mini Spray Dryer (BÜCHI Labortechnik AG, Flawil, Switzerland), ProCepT Formatrix R&D spray dryer (ProCepT nv, Zelzate, Belgium), among several other spray dryer options. Actual spray drying conditions will vary depending, in part, on the composition of the spray drying solution or suspension and material flow rates. The person of ordinary skill will be able to determine appropriate conditions based on the compositions of the solution, emulsion or suspension to be spray dried, the desired particle properties and other factors. In general, the inlet temperature to the spray dryer is about 90 ° C to about 300 °C The spray dryer outlet temperature will vary depending upon such factors as the feed temperature and the properties of the materials being dried. Generally, the outlet temperature is about 50 ° C to about 150 °C If desired, the respirable dry particles that are produced can be fractionated by volumetric size, for example, using a sieve, or fractioned by aerodynamic size, for example, using a cyclone, and/or further separated according to density using techniques known to those of skill in the art.

To prepare the respirable dry particles, generally, an emulsion or suspension that contains the desired components of the dry powder (i.e., a feedstock) is prepared and spray dried under suitable conditions. Preferably, the dissolved or suspended solids concentration in the feedstock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L or at least about 100 g/L. The feedstock can be provided by preparing a single solution, suspension or emulsion by dissolving, suspending, or emulsifying suitable components (e.g., salts, excipients, other active ingredients) in a suitable solvent. The solution, emulsion or suspension can be prepared using any suitable methods, such as bulk mixing of dry and/or liquid components or static mixing of liquid components to form a combination. For example, a hydrophilic component (e.g., an aqueous solution) and a hydrophobic component (e.g., an organic solution) can be combined using a static mixer to form a combination. The combination can then be atomized to produce droplets, which are dried to form respirable dry particles. Preferably, the atomizing step is performed immediately after the components are combined in the static mixer. Alternatively, the atomizing step is performed on a bulk mixed solution.

The feedstock can be prepared using any solvent in which the itraconazole in particulate form has low solubility, such as an organic solvent, an aqueous solvent or mixtures thereof. Suitable organic solvents that can be employed include but are not limited to alcohols such as, for example, ethanol, methanol, propanol, isopropanol, butanols, and others. Other organic solvents include but are not limited to tetrahydrofuran (THF), perfluorocarbons, dichloromethane, chloroform, ether, ethyl acetate, methyl tert-butyl ether and others. Co-solvents that can be employed include an aqueous solvent and an organic solvent, such as, but not limited to, the organic solvents as described above. Aqueous solvents include water and buffered solutions. A preferred solvent is water.

Various methods (e.g., static mixing, bulk mixing) can be used for mixing the solutes and solvents to prepare feedstocks, which are known in the art. If desired, other suitable methods of mixing may be used. For example, additional components that cause or facilitate the mixing can be included in the feedstock. For example, carbon dioxide produces fizzing or effervescence and thus can serve to promote physical mixing of the solute and solvents.

The feedstock or components of the feedstock can have any desired pH, viscosity or other properties. If desired, a pH buffer can be added to the solvent or co-solvent or to the formed mixture. Generally, the pH of the mixture ranges from about 3 to about 8.

Dry powder and/or respirable dry particles can be fabricated and then separated, for example, by filtration or centrifugation by means of a cyclone, to provide a particle sample with a preselected size distribution. For example, greater than about 30%, greater than about 40%, greater than about 50%, greater than about 60%, greater than about 70%, greater than about 80%, or greater than about 90% of the respirable dry particles in a sample can have a diameter within a selected range. The selected range within which a certain percentage of the respirable dry particles fall can be, for example, any of the size ranges described herein, such as between about 0.1 to about 3 microns VMGD.

The suspension may be a nano-suspension, similar to an intermediate for making dry powder comprising nano-crystalline itraconazole.

The dry powder may be itraconazole embedded in a matrix material, such as a matrix material comprising sodium sulfate and leucine. Optionally, the dry powder may be spray dried such that the dry particles are small, dense, and dispersible.

The dry powders can consist solely of the respirable dry particles described herein without other carrier or excipient particles (referred to as “neat powders”). If desired the dry powders can comprise blends of the respirable dry particles described herein and other carrier or excipient particles, such as lactose carrier particles that are greater than 10 microns, 20 microns to 500 microns, and preferably between 25 microns and 250 microns. In some embodiments, dry powders comprising carrier particles (blended powders) are excluded.

In a preferred embodiment, the dry powders do not comprise carrier particles. In one aspect, the itraconazole is embedded in a matrix comprising a sodium salt, leucine, and stabilizer. The dry powder may comprise respirable dry particles of uniform content, wherein each particle comprises the itraconazole. Thus, as used herein, “uniform content” means that every respirable particle comprises some amount of itraconazole, with the stabilizer, sodium salt, and leucine.

The dry powders can comprise respirable dry particles wherein at least 98%, at least 99%, or substantially all of the particles (by weight) comprise itraconazole.

The dry powders are typically manufactured by first processing the itraconazole in crystalline form to adjust the particle size using any number of techniques that are familiar to those of skill in the art (e.g., wet milling, jet milling). For example, crystalline itraconazole may be processed in an antisolvent with a stabilizer to form a suspension. Preferred stabilizers include polysorbates (also known as TWEEN®), such as polysorbate 80 (PS80). The stabilized suspension of crystalline itraconazole is then spray dried with the sodium salt and leucine. The resulting dry particles comprise crystalline itraconazole dispersed throughout an excipient matrix with each dry particle having a homogenous composition.

In a particular embodiment, a dry powder of the present invention is made by starting with crystalline itraconazole, which is usually obtainable in a micro-crystalline size range. The particle size of the micro-crystalline itraconazole is reduced into the nano-crystalline size using any of a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain. What becomes especially important in the sub-micron size domain is the use of surface stabilizing components, such as surfactants (e.g., polysorbate 80, also known as TWEEN® 80). Surfactants enable the creation of submicron particles during milling and the formation of physically stable suspensions, as they sequester the many high energy surfaces created during milling preventing aggregation and sedimentation. Thus, the presence of the surfactant is important to spray drying homogenous micro-particles as the surfactant allows for the formation of a uniform and stable suspension ensuring compositional homogeneity across particles. The use of surfactant allows for formation of micro-suspension or nano-suspensions. With the surfactant, the nano-crystalline itraconazole particles are suspended in a stable colloidal suspension in the anti-solvent. The anti-solvent for the drug can utilize water, or a combination of water and other miscible solvents such as alcohols or ketones as the continuous anti-solvent phase for the colloidal suspension. A spray drying feedstock may be prepared by dissolving the soluble components in a desired solvent(s) followed by dispersing the surfactant-stabilized crystalline itraconazole nanosuspension in the resulting feedstock while mixing, although the process is not limited to this specific order of operations.

Methods for analyzing the dry powders and/or respirable dry particles are found in the Exemplification section below.

### Exemplification

Materials used in the following Examples and their sources are listed below. Sodium sulfate, polysorbate 80, and L-leucine were obtained from Sigma-Aldrich Co. (St. Louis, MO), Spectrum Chemicals (Gardena, CA), Applichem (Maryland Heights, MO), Alfa Aesar (Tewksbury, MA), Thermo Fisher (Waltham, MA), Croda Chemicals (East Yorkshire, United Kingdom) or Merck (Darmstadt, Germany). Itraconazole was obtained from Neuland (Princeton, NJ). Ultrapure (Type II ASTM) water was from a water purification system (Millipore Corp., Billerica, MA), or equivalent.

PBMK studies were performed in silico. The methods and results of a Phase 1 clinical study using an exemplary respirable dry powder used for static equation calculation and model development are summarized in Hava et al. (supra).

Geometric or Volume Diameter: Volume median diameter (x50 or Dv50), which may also be referred to as volume median geometric diameter (VMGD), of the dry powders was determined using a laser diffraction technique. The equipment consisted of a HELOS diffractometer and a RODOS dry powder disperser (Sympatec, Inc., Princeton, NJ). The RODOS disperser applies a shear force to a sample of particles, controlled by the regulator pressure (typically set at 1.0 bar with maximum orifice ring pressure) of the incoming compressed dry air. The pressure settings may be varied to vary the amount of energy used to disperse the powder. For example, the dispersion energy may be modulated by changing the regulator pressure from 0.2 bar to 4.0 bar. Powder sample is dispensed from a microspatula into the RODOS funnel. The dispersed particles travel through a laser beam where the resulting diffracted light pattern produced is collected, typically using an R1 lens, by a series of detectors. The ensemble diffraction pattern is then translated into a volume-based particle size distribution using the Fraunhofer diffraction model, on the basis that smaller particles diffract light at larger angles. Using this method, the span of the distribution was also determined per the formula ((Dv[90]−Dv[10))/(Dv[50]). The span value gives a relative indication of the polydispersity of the particle size distribution.

Aerodynamic Performance: The aerodynamic properties of the powders dispersed from an inhaler device were assessed with a Next Generation Impactor (Copley Scientific Limited, Nottingham, UK) (NGI). For measurements utilizing the NGI, the NGI instrument was run in controlled environmental conditions of 18 to 25 ° C and relative humidity (RH) between 25 and 35%. The instrument consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Gravimetric and/or chemical analyses can then be performed to determine the particle size distribution. The capsules (HPMC, Size 3; Capsugel Vcaps, Peapack, NJ) were filled with powder to a specific weight and placed in a hand-held, breath-activated dry powder inhaler (DPI) device, the high resistance RS01 DPI or the ultra-high resistance RS01 DPI (both by Plastiape, Osnago, Italy). The capsule was punctured and the powder was drawn through the cascade impactor operated at a specified flow rate for 2.0 Liters of inhaled air. At the specified flow rate, the cut-off diameters for the stages were calculated. The fractions were collected by placing wetted filters in the apparatus and determining the amount of powder that impinged on them by chemical measurements on an HPLC.

Fine Particle Dose: The fine particle dose indicates the mass of itraconazole in a specific size range and can be used to predict the mass which will reach a certain region in the respiratory tract. The fine particle dose can be measured gravimetrically or chemically via either an ACI or NGI. If measured gravimetrically, since the dry particles are assumed to be homogenous, the mass of the powder on each stage and collection filter can be multiplied by the fraction of itraconazole in the formulation to determine the mass of itraconazole. If measured chemically, the powder from each stage or filter is collected, separated, and assayed for example on an HPLC to determine the content of the itraconazole. The cumulative mass deposited on each of the stages at the specified flow rate is calculated and the cumulative mass corresponding to a 5.0 micrometer diameter particle is interpolated. This cumulative mass for a single dose of powder, contained in one or more capsules, actuated into the impactor is equal to the fine particle dose less than 5.0 microns (FPD<5.0 microns).

Mass Median Aerodynamic Diameter (MMAD): MMAD was determined using the information obtained by the Next Generation Impactor (NGI). The cumulative mass under the stage cut-off diameter is calculated for each stage and normalized by the recovered dose of powder. The MMAD of the powder is then calculated by linear interpolation of the stage cut-off diameters that bracket the 50th percentile. An alternative method of measuring the MMAD is with an Andersen Cascade Impactor (ACI). Like the NGI, the MMAD is calculated with the cumulative mass under the stage cut-off diameter is calculated for each stage and normalized by the recovered dose of powder. The MMAD of the powder is then calculated by linear interpolation of the stage cut-off diameters that bracket the 50th percentile.

Emitted Geometric or Volume Diameter: The volume median diameter (Dv50) of the powder after it is emitted from a dry powder inhaler, which may also be referred to as volume median geometric diameter (VMGD), was determined using a laser diffraction technique via the Spraytec diffractometer (Malvern, Inc.). Powder was filled into size 3 capsules (V-Caps, Capsugel) and placed in a capsule based dry powder inhaler (RS01TM Model 7 High resistance, Plastiape, Italy), or DPI, and the DPI sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve. A steady air flow rate was drawn through the DPI typically at 60 L/min for a set duration, typically of 2 seconds. Alternatively, the air flow rate drawn through the DPI was sometimes run at 15 L/min, 20 L/min, or 30 L/min. The resulting geometric particle size distribution of the aerosol was calculated from the software based on the measured scatter pattern on the photodetectors with samples typically taken at 1000 Hz for the duration of the inhalation. The Dv50, GSD, FPF<5.0 μm measured were then averaged over the duration of the inhalation.

The Emitted Dose (ED) refers to the mass of itraconazole which exits a suitable inhaler device after a firing or dispersion event. The ED is determined using a method based on USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, MD, 13th Revision, 222-225, 2007. Contents of capsules are dispersed using either the RS01 HR inhaler at a pressure drop of 4 kPa and a typical flow rate of 60 LPM or the UHR2 RS01 at a pressure drop of 4 kPa and a typical flow rate of 39 LPM. The emitted powder is collected on a filter in a filter holder sampling apparatus. The sampling apparatus is rinsed with a suitable solvent such as water and analyzed using an HPLC method. For gravimetric analysis a shorter length filter holder sampling apparatus is used to reduce deposition in the apparatus and the filter is weighed before and after to determine the mass of powder delivered from the DPI to the filter. The emitted dose of therapeutic is then calculated based on the content of therapeutic in the delivered powder. Emitted dose can be reported as the mass of therapeutic delivered from the DPI or as a percentage of the filled dose.

Thermogravimetric Analysis: Thermogravimetric analysis (TGA) was performed using either the Q500 model or the Discovery model thermogravimetric analyzer (TA Instruments, New Castle, DE). The samples were either placed into an open aluminum DSC pan or a sealed aluminum DSC pan that was then automatically punched open prior to the time of test. Tare weights were previously recorded by the instrument. The following method was employed: Ramp 5.00 ° C/min from ambient (~35 ° C) to 200 °C The weight loss was reported as a function of temperature up to 140 °C TGA allows for the calculation of the content of volatile compounds within the dry powder. When utilizing processes with water alone, or water in conjunction with volatile solvents, the weight loss via TGA is a good estimate of water content.

X-Ray Powder Diffraction: The crystalline character of the formulations was assessed via powder X-ray diffraction (PXRD). A 20-30 mg sample of material is analyzed in a powder X-ray diffractometer (D8 Discover with LINXEYE detector; Bruker Corporation, Billerica, MA or equivalent) using a Cu X-ray tube with 1.5418A at a data accumulation time 1.2 second/step over a scan range of 5 to 45° 2θ and a step size of 0.02° 2θ.

Itraconazole Content/Purity using HPLC: A high performance liquid chromatography (HPLC) method utilizing a reverse phase C18 column coupled to an ultraviolet (UV) detector has been developed for the identification, bulk content, assay, CUPMD and impurities analysis of itraconazole dry powders. The reverse phase column is equilibrated to 30 ° C and the autosampler is set to 5 °C The mobile phases, 20 mM sodium phosphate monobasic at a pH of 2.0 (mobile phase A) and acetonitrile (mobile phase B) are used in a gradient elution from a ratio of 59:41 (A:B) to 5:95 (A:B), over the course of a 19.5 minute run time. Detection is by UV at 258 nm and the injection volume is 10 μL. Itraconazole content in powders are quantified relative to a standard curve.

Identification of known impurities A, B, C, D, E, F and G (shown in monograph Ph. Eur. 01/2011:1335) is confirmed by comparing the retention time of the impurity peaks in the itraconazole dry powder samples to that of the itraconazole USP impurity mix reference standard spiked with impurity A. Unknown impurities are identified and quantified by relative retention time to that of the itraconazole main peak and with area above the limit of detection (LOD). All impurities are measured by area percent, with respect to the itraconazole peak.

Particle Size Reduction: The particle size distribution of the crystalline itraconazole can be modulated using a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain.

Particle Size Reduction using Low Energy Wet Milling. One technique for reducing the particle size of the itraconazole was via low energy wet milling (also known as roller milling, or jar milling). Suspensions of the itraconazole were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The vessels containing the suspensions are then rotated using a jar mill (US Stoneware, East Palestine, OH USA) while taking samples periodically to assess particle size (LA-950, HORIBA, Kyoto, Japan). When the particle size is sufficiently reduced, or when a particle size minimum is reached, the suspension is strained through a sieve to remove the milling media, and the product recovered.

Particle Size Reduction using High Energy Wet Milling: Another technique for reducing the particle size of the itraconazole was via high-energy wet milling using a rotor-stator, or agitated media mill. Suspensions of the itraconazole were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The suspensions are then charged into the mill, which can be operated in either batch or recirculation mode. The process consists of the suspension and milling media being agitated within the milling chamber, which increases the energy input to the system and accelerates the particle size reduction process. The milling chamber and recirculation vessel are jacketed and actively cooled to avoid temperature increases in the product. The agitation rate and recirculation rate of the suspension are controlled during the process. Samples are taken periodically to assess particle size (LA-950, HORIBA, Kyoto, Japan). When the particle size is sufficiently reduced, or when a particle size minimum is reached, the suspension is discharged from the mill.

Spray Drying: Dry powders were prepared by spray drying on a Büchi B-290 Mini Spray Dryer (BÜCHI Labortechnik AG, Flawil, Switzerland) with powder collection from either a standard or High Performance cyclone. The system was run with nitrogen as the drying and atomization gas in open-loop (single pass) mode. When run using air, the system used the Büchi B-296 dehumidifier to ensure stable temperature and humidity of the air used to spray dry. Furthermore, when the relative humidity in the room exceeded 30% RH, an external LG dehumidifier (model 49007903, LG Electronics, Englewood Cliffs, NJ) was run constantly. When run using nitrogen, a pressurized source of nitrogen was used. Furthermore, the aspirator of the system was adjusted to maintain the system pressure at −2.0″ water column. Atomization of the liquid feed utilized a Büchi nozzle with 1.5 mm cap and 0.7 liquid tip. The liquid feedstock solids concentration was 3%, the process gas inlet temperature was 127 ° C to 140 ° C, the process gas outlet temperature was 60 ° C, the drying gas flowrate was 17.0 kg/hr, the atomization gas flowrate was 30.0 g/min, and the liquid feedstock flowrate was 6.0 mL/min.

Stability Assessment: The physicochemical stability and aerosol performance of select formulations were assessed at 2-8 ° C, 25 ° C/60% RH, and when material quantities permitted, 40 ° C/75% RH as detailed in the International Conference on Harmonisation (ICH) Q1 guidance. Stability samples were stored in calibrated chambers (Darwin Chambers Company Models PH024 and PH074, St. Louis. MO). Bulk powder samples were weighed into amber glass vials, sealed under 30% RH, and induction-sealed in aluminum pouches (Drishield 3000, 3M, St. Paul, MN) with silica desiccant (2.0 g, Multisorb Technologies, Buffalo, NY). Additionally, to assess the stability of the formulations in capsules, the target mass of powder was weighed by hand into a size 3, HPMC capsule (Capsugel Vcaps, Peapack, NJ) with a +/−0.2 mg tolerance at 30% RH. Filled capsules were then aliquoted into high-density polyethylene (HDPE) bottles and induction sealed in aluminum pouches with silica desiccant.

### Example 1: Preparation and Characterization of Exemplary Dry Powder Formulation I A. Powder Preparation

The nanocrystalline itraconazole for Formulation I was prepared by compounding 30.090 g of itraconazole (Neuland ITI0114005 and ITI0714011) in 87 g of water and 3 g of polysorbate 80. Polystyrene milling media (130 g of 500 μm; Dow Chemical, Midland MI) was then added to the suspension, and the suspension was milled at 1800 rpm for one hour before being collected. The final median particle size (Dv(50)) of the milled suspension was 132 nm.

A feedstock solution was then prepared and used to manufacture the dry powder. A drug load of 50 wt % itraconazole, on a dry basis, was targeted. The feedstock solution that was used to spray dry particles were made as follows. The required quantity of water (1.18 kg) was weighed into a suitably sized glass vessel. Sodium sulfate (12.8 g) and leucine (3.7 g) were added to the water and the solution allowed to stir until visually clear. The itraconazole-containing suspension (containing 18.3 grams itraconazole and 1.83 grams polysorbate 80) was then added to the excipient solution and stirred until visually homogenous. The feedstock was then spray-dried. Feedstocks were stirred while spray dried. Feedstock mass was approximately 1.22 kg. Dry powders Formulation I was manufactured from the feedstock by spray drying on the Büchi B-290 Mini Spray Dryer (BÜCHI Labortechnik AG, Flawil, Switzerland) with cyclone powder collection, following the protocol described above.

Formulation I has the following dry powder composition (w/w) on a dry basis: 50% itraconazole, 35% sodium sulfate, 10% leucine, and 5% polysorbate 80.

### B. Powder Characterization.

The bulk particle size characteristics for Formulation I is provided below in Table 2. The span at 1 bar of less than 2.10 indicates a relatively narrow size distribution. The 1 bar/4 bar dispersibility ratio less than 1.25 indicates the powder is relatively independent of dispersion energy, a desirable characteristic which allows similar particle dispersion across a range of dispersion energies.

TABLE 2
Bulk particle size characteristics of Formulation I

| | 0.5 bar | | 1 bar | | 4 bar | | |
|---|---|---|---|---|---|---|---|
| | Dv[50] | | Dv[50] | | Dv[50] | | 1 bar:4 bar |
| | (μm) | Span | (μm) | Span | (μm) | Span | Dv[50] ratio |
| Formu- | 3.61 | 2.01 | 3.46 | 2.08 | 3.05 | 2.15 | 1.14 |
| lation I | | | | | | | |

The geometric particle size and capsule emitted powder mass (CEPM) measured and/or calculated at 60 liters per minute (LPM) and 30 LPM simulated patient flow rates was also measured for Formulation I. At 30 LPM, Formulation I had a CEPM of 99.3% and Dv50 of 4.35 μm. At 60 LPM, Formulation I had a CEPM of 99.8% and Dv50 of 3.97 μm. The small changes in CEPM and geometric size from 60 LPM to 30 LPM indicates that the dry powder is relatively independent of patient inspiratory flowrate, indicating that patients breathing in at varying flow rates would receive a relatively similar therapeutic dose.

The aerodynamic particle size, fine particle fractions and fine particle doses were also measured and/or calculated with a Next Generation Impactor (NGI). Formulation I had a MMAD of 4.22 μm, and an FPD<5 μm of 38.3% of nominal dose. In other words, more than 30% of the nominal dose reaches the impactor stages and so would be predicted to be delivered to the lungs. The MMAD of 4.22 is also indicative of deposition in the central and conducting airways.

Thermogravimetric analysis determined the weight loss was 0.1%.

The crystallinity of Formulation I was assessed via X-ray diffraction (XRD). The diffraction pattern of itraconazole is observed in the formulation, suggesting the milling or spray drying process did not affect the solid-state of itraconazole.

### Example 2: In Silico Modelling

A physiological-based pharmacokinetics (PBPK) model originally developed to simulate the concentration-time profiles of itraconazole and OH-itraconazole after administration of itraconazole as an oral solution (Simcyp Simulator, V19) was modified to simulate administration by oral inhalation. The PBPK model parameters describing the fraction absorbed and rate of absorption from the lung, as well as the fraction of inhaled dose swallowed, were optimized by fitting these parameters to the observed plasma concentration data collected in a clinical study in which 35 mg of Formulation I was administered once a day (QD) for 14 days. Simulated population geometric mean itraconazole and OH-itraconazole area under the curve (AUC) 0-24 h values at steady state (by Day 14) were within 0.96-fold and 1.68-fold respectively of observed data from a Phase 1 clinical trial using Formulation I. See Hava, supra). The data could be used to determine the DDI potential of a maximum 40 mg dose of orally inhaled itraconazole, by applying basic static model, a mechanistic static model, and a physiologically based pharmacokinetic (PBPK) model of itraconazole and OH-itraconazole, the primary metabolite of itraconazole, to evaluate the potential risk of itraconazole dry powders as “perpetrators” of CYP3A4 DDIs, using midazolam as a “victim” drug and Formulation I as an exemplary itraconazole based dry powder.

Basic Static Model of Reversible Inhibition: The basic and mechanistic static equations have been described in the FDA guidance for in vitro drug interaction studies (FDA DDI Guidance (2020). “Clinical Drug Interaction Studies-Cytochrome P450 Enzyme- and Transporter-Mediated Drug Interactions, Guidance for Industry.” U.S. Department of Health and Human Services, Food and Drug Administration, Center for Drug Evaluation and Research (CDER); incorporated herein by reference in its entirety).

For Formulation I, the ratio of the intrinsic clearance value of a probe substrate in the absence and in the presence of itraconazole (R1) was calculated using the observed Cₘₐₓ of itraconazole and OH-itraconazole, respectively, after 14 days of 35 mg of Formulation I QD.

Calculation of the ratio of the intrinsic clearance value of a probe substrate in the absence and in the presence of an inhibitor in the gut (R1,gut) was used for Formulation I, as itraconazole and OH-itraconazole are inhibitors of CYP3A4. Equation input values are listed in Table 3.

TABLE 3
Parameters used for the calculation of R values for
the static equations of reversible inhibition.

| Parameter | Value | Source |
|---|---|---|
| Itraconazole | | |
| Molecular weight | 705.6 | Simcyp V19 Itraconazole File |
| (g/mol) | | |
| fᵤ | 0.016 | Simcyp V19 Itraconazole File |
| R_{B} | 0.58 | Simcyp V19 Itraconazole File |
| Ki, u, ITZ (μM) | 0.0013 | Simcyp V19 Itraconazole File |
| OH-itraconazole | | |
| Molecular weight | 721.7 | Simcyp V19 OH-itraconazole File |
| (g/mol) | | |
| fᵤ | 0.016 | Simcyp V19 OH-itraconazole File |
| R_{B} | 0.58 | Simcyp V19 OH-itraconazole File |
| Ki, u, ITZ (μM) | 0.0023 | Simcyp V19 OH-itraconazole File |
| Formulation I | | |
| Dose (mg) | 35 | Hava (supra) |
| Fₐ | 0.159 | Hava (supra) |
| Cmax, ITZ (μM) | 0.0215 | Hava (supra) day 14 Cₘₐₓ, converted to μM from |
| | | ng/mL |
| Cmax, OH-ITZ (μM) | 0.0120 | Simcyp V19 OH-itraconazole File day 14 Cₘₐₓ, |
| | | converted to μM from ng/mL |
| Midazolam | | |
| Fₐ | 1 | Simcyp V19 Midazolam File |
| F_{g} | 0.58 | Simcyp V19 Midazolam File |
| fₘ | 0.84 | Simcyp V19 Midazolam File |
| Biological parameters | | |
| Qₑₙ (L/hr/70 kg | 19 | FDA DDI Guidance 2020 |
| Qₕ (L/hr/70 kg) | 97 | FDA DDI Guidance 2020 |

An R1≥1.02 or R1,gut ≥11 indicates that a clinically significant drug-drug interaction may exist and requires further investigation.

Mechanistic Static Model of Reversible Inhibition: The area under the plasma concentration-time curve ratio (AUCR) for reversible inhibitors was calculated according to the FDA guidance for in vitro drug interaction studies. As a worst-case scenario, it was assumed that all of the inhaled dose was swallowed and subsequently delivered to the gut. A weak, moderate, or strong DDI is defined as a calculated AUCR (weak: ≥1.25-fold, but <2.00-fold; moderate: ≥2.00-fold, but <5.00-fold; strong: ≥5.00-fold).

PBPK Modelling-Study Design and Setup: To achieve the modelling objectives, this step in the investigation consisted of two parts, model optimization and application. Some of the key factors relating to each step are indicated below. Model optimization and application were performed using Simcyp (2019, Version 19 Release 2, Certara, Sheffield, UK). Data assembly and plotting were performed using RStudio (Version 4.1.2; R Foundation for Statistical Computing, Vienna, Austria). The virtual North European Caucasian population (physiological parameters including liver volume and blood flows, enzyme abundances) within Simcyp was used for all simulations (Howgate E., et al., Xenobiotica (2006) 36 (6): 473-497). Except for demographic data, all parameter values for the healthy volunteer (HV) population are the same as those used for the Caucasian population.

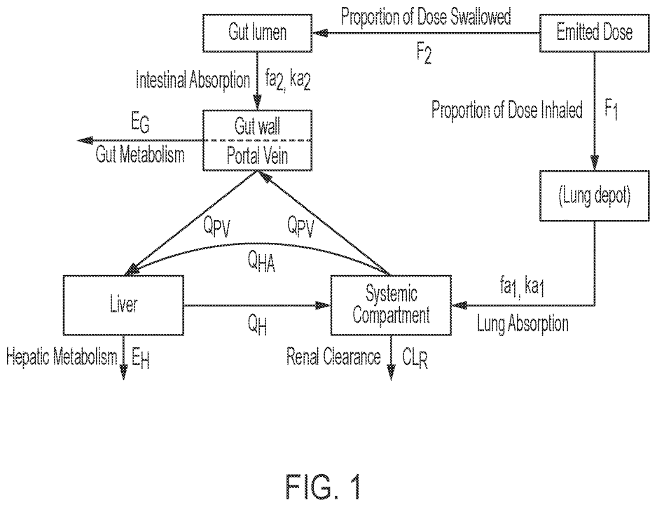

PBPK Modelling-Model Optimization: To simulate the plasma concentration-time profiles of itraconazole and OH-itraconazole after the inhaled administration of 35 mg Formulation I (QD for 14 days), the Simcyp V19 itraconazole oral solution PBPK model was adapted to include absorption of itraconazole through the lung as well as the gut. The model assumes first-order absorption from the lungs to the systemic circulation. The structural model is shown in FIG. 1. The trial design used for the optimization of first-order inhalation parameters was based on the study described in Hava et al. (supra) in which subjects received Formulation I at 35 mg QD for 14 days. That study measured itraconazole and OH-itraconazole data as summarized in Table 4.

TABLE 4
First order inhalation parameters for itraconazole and OH-itraconazole using Formulation I.

| | Tₘₐₓ | Cₘₐₓ | AUC_{0-24 h} | | Accumulation | Accumulation |
|---|---|---|---|---|---|---|
| Dose (mg) | (h) | (ng/mL) | (ng h/mL) | Kel (1/h) | ratio Cₘₐₓ | ratio AUC_{0-24 h} |
| Itraconazole | | | | | | |
| 10 | 5 | 3.77 (34.2) | 73.2 (35.1) | 0.0169 | 2.9 | 3.0 |
| 20 | 4 | 8.98 (37.9) | 175 (32.7) | 0.0197 | 3.4 | 3.3 |
| 35 | 0.75 | 15.2 (49.3) | 276 (62.2) | 0.0170 | 3.0 | 2.8 |
| OH-itraconazole | | | | | | |
| 10 | 6 | 2.25 (25.3) | 42.4 (26.1) | 0.0263 | 3.5 | 3.8 |
| 20 | 6 | 6.43 (54.7) | 128 (56.1) | 0.0223 | 4.2 | 4.4 |
| 35 | 8 | 8.68 (91.0) | 169 (116) | 0.0248 | 4.2 | 4.5 |

For the PBPK model development, ten virtual trials of six subjects, aged 21 to 58 years (33.3% female), were generated to assess variability across groups. The population of the virtual trial was selected to match the clinical study subjects. A range of values was tested for the proportion of dose inhaled, the fraction of itraconazole absorbed from the lung (Fa, 1), and the first-order rate constant of itraconazole absorption from the lung (ka, 1). These parameters were optimized to best fit the observed plasma concentration-time profiles and PK parameters of itraconazole and OH-itraconazole after multiple dose administration of Formulation I on study day 14, after 35 mg Formulation I (QD for 14 days). Briefly, the pro-portion of dose inhaled and Fa, 1 was optimized to match the itraconazole AUC0-24 h on study day 14. Then, ka, 1 was optimized to capture Cₘₐₓ. Once the simulated multiple dose and Cₘₐₓ were within 0.8 to 1.25 of the observed values, the absorption parameters were further optimized to best predict the OH-itraconazole concentration-time profiles. Intestinal absorption, distribution, and elimination parameters of itraconazole and OH-itraconazole were unchanged from the verified itraconazole and OH-itraconazole models, assuming itraconazole follows linear kinetics even at low doses. This model assumes no metabolism of itraconazole to OH-itraconazole in the lung.

The first-order inhalation parameters were manually optimized by comparing the simulated profiles of Formulation I to observed data. The first-order inhalation parameters used to simulate the plasma concentration profile of Formulation I are shown in Table 5.

TABLE 5
Final input optimized absorption parameters of Formulation I.

| Parameter | Formulation I | Reference |
|---|---|---|
| Proportion of | 99.9 | Manually optimized |
| dose inhaled (%) | | |
| F_{a, 1} | 0.265 | Manually optimized |
| K_{a, 1} (1/hr) | 0.028 | Manually optimized |

PBPK Modelling-Model Application: The CYP3 A4 inhibition potential of Formulation I was predicted using a representative virtual healthy population consisting of ten virtual trials of ten healthy subjects (50% female), aged 20 to 50 years. The victim drug (midazolam) was administered as a single dose of 5 mg without administration of Formulation I, then again on day 14 of daily administration of Formulation I 35 mg. The virtual DDI trials were repeated with the same virtual trial design and study population, with 40 mg of Formulation I administered daily for 14 days.

Calculation of the R Value for the Basic Model of Reversible Inhibition: R1 was calculated for Formulation I using the observed maximum concentration of itraconazole and OH-itraconazole following multiple inhaled doses of Formulation I (35 mg QD) for 14 days. R1 was calculated to be 1.35. Because this value exceeded the cutoff value of 1.02 specified by the FDA guidance document (supra), further investigation of DDI liability was required. Additionally, R1,gut was calculated to be greater than the threshold value of 11, indicating that additional assessment of DDI liability was required. Based on these results, a mechanistic static model was applied to further investigate the CYP3A4 inhibition potential of Formulation I.

Calculation of AUCR for the Mechanistic Static Equation: AUCR of midazolam was calculated for Formulation I using the observed maximum concentration of itraconazole and OH-itraconazole following multiple simulated inhaled doses of Formulation I (35 mg QD) for 14 days. To calculate the worst-case scenario, it was assumed that all itraconazole absorption occurred through the gut. The AUCR of midazolam was calculated to be 5.36, further indicating a risk of Formulation I as a perpetrator of CYP3A4 DDIs. Based on these results, a PBPK model was developed to further understand the CYP3A4 inhibition potential of Formulation I.

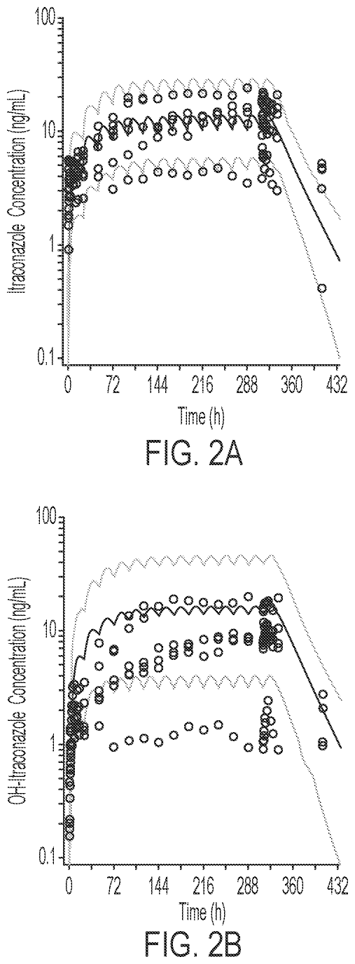

PBPK Model Optimization and Application: Simulated itraconazole and OH-itraconazole plasma concentration data are based on the manually optimized proportion of dose inhaled, (Fa, 1), and (ka, 1) parameters of Formulation I. Individual mean trial concentrations and the mean concentration-time profiles for the total virtual population (n=60) were simulated. The simulated profiles of Formulation I and OH-itraconazole after 14 days of dosing with 35 mg/day of Formulation I were comparable to the clinical data as shown in FIGS. 2A and 2B. In addition, the predicted geometric mean Cₘₐₓ and AUC0-24 h values for itraconazole on day 14 were within 0.81- and 0.96-fold, respectively, of the observed values (Table 6).

TABLE 6
Predicted and Observed Itraconazole Cₘₐₓ and AUC_{0-24 h} After
Multiple 35 mg Inhaled Doses of Formulation I (QD 14 Days).

| | Day 1 | | Day 14 | |
|---|---|---|---|---|
| | Cₘₐₓ | AUC_{0-24 h} | Cₘₐₓ | AUC_{0-24 h} |
| | (ng/mL) | (h · ng/mL) | (ng/mL) | (h · ng/mL) |
| Simulated | | | | |
| Geometric | 4.93 | 97.4 | 12.3 | 266 |
| mean | | | | |
| CV % | 49 | 48 | 51 | 53 |
| 5^{th} percentile | 1.81 | 35.6 | 28.8 | 627 |
| 95^{th} percentile | 9.74 | 197 | 28.8 | 627 |
| Observed | | | | |
| Mean | 5.23 | 102 | 15.2 | 276 |
| CV % | 26 | 24 | 49 | 62 |
| S/O | 0.94 | 0.95 | 0.81 | 0.96 |

The predicted mean Cₘₐₓ and AUC0-24 h values for OH-itraconazole on day 14 were within 1.47- and 1.68-fold, respectively, of the observed values (Table 7).

TABLE 7
Predicted and Observed OH-itraconazole Cₘₐₓ and AUC_{0-24 h} After
Multiple 35 mg Inhaled Doses of Formulation I (QD 14 Days).

| | Day 1 | | Day 14 | |
|---|---|---|---|---|
| | Cₘₐₓ | AUC_{0-24 h} | Cₘₐₓ | AUC_{0-24 h} |
| | (ng/mL) | (h · ng/mL) | (ng/mL) | (h · ng/mL) |
| Simulated | | | | |
| Geometric | 4.9 | 83.9 | 12.8 | 284 |
| mean | | | | |
| CV % | 74 | 67 | 84 | 87 |
| 5^{th} percentile | 1.68 | 27.0 | 3.99 | 87.3 |
| 95^{th} percentile | 15.8 | 256 | 45.8 | 1025 |
| Observed | | | | |
| Mean | 2.10 | 38.2 | 8.68 | 168 |
| CV % | 36 | 34 | 91 | 116 |
| S/O | 2.33 | 2.19 | 1.47 | 1.68 |

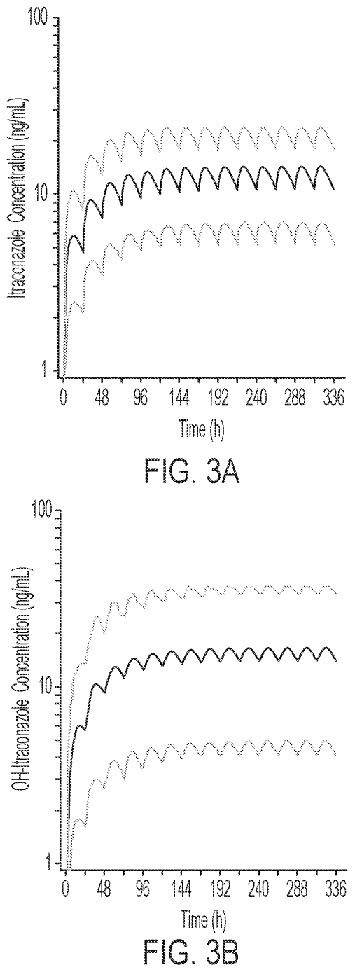

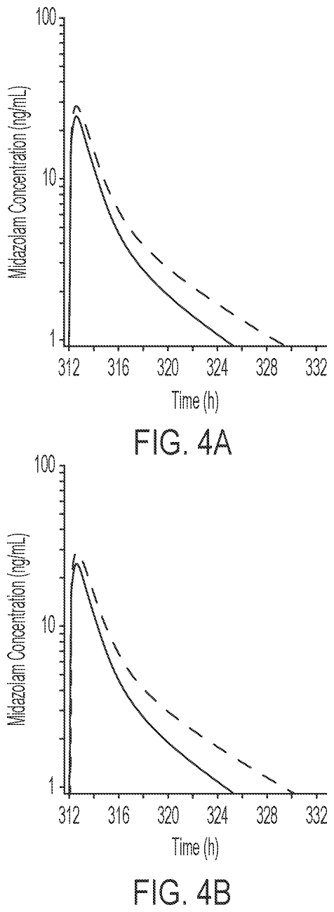

For model application, plasma concentration-time profiles of midazolam following a single oral dose of 5 mg in the absence of Formulation I and on the 14th day of 14 days of dosing of Formulation I (35 mg daily or 40 mg daily) to healthy subjects were simulated. The mean simulated plasma concentrations of itraconazole and OH-itraconazole after 14 days of 40 mg daily dosing of Formulation I are presented in FIGS. 3A and 3B. Mean simulated plasma midazolam concentrations following a single oral dose of 5 mg in the absence of Formulation I and on the 14th day of 14 days of dosing of Formulation I (35 mg daily or 40 mg daily) to healthy subjects are shown in FIGS. 4A and 4B. The predicted geometric mean Cₘₐₓ and AUC_{0-inf} values and corresponding geometric mean ratios for midazolam in the presence and absence of Formulation I are shown in Table 8. The threshold for a weak DDI (AUCR and CₘₐₓR <1.25) was not met when midazolam was coadministered with 35 mg daily of Formulation I. At the higher dose of 40 mg daily Formulation I, a weak DDI is predicted (AUCR≥1.25 but <2).

TABLE 8
Predicted geometric mean Cₘₐₓ and AUC_{0-inf} values and corresponding
geometric mean ratios for midazolam in the absence and presence of
Formulation I (35 mg or 40 mg QD for 14 days) in healthy subjects.

| | Midazolam | | Midazolam + Formulation I | | | |
|---|---|---|---|---|---|---|
| | AUC_{0-inf} | Cₘₐₓ | AUC_{0-inf} | Cₘₐₓ | GMR | |
| | (h · ng/mL) | (ng/mL) | (h · ng/mL) | (ng/mL) | AUC_{0-inf} | Cₘₐₓ |
| Formulation I | | | | | | |
| 35 mg QD, 14 days | | | | | | |
| Geometric mean | 58.4 | 19.3 | 71.4 | 21.6 | 1.22 | 1.12 |
| 90% CI-lower | 51.3 | 17.2 | 61.8 | 19.2 | 1.20 | 1.11 |
| 90% CI-upper | 66.5 | 21.6 | 82.6 | 24.3 | 1.25 | 1.13 |
| Formulation I | | | | | | |
| 40 mg QD, 14 days | | | | | | |
| Geometric mean | 58.4 | 19.3 | 70.4 | 21.9 | 1.26 | 1.14 |
| 90% CI-lower | 51.3 | 17.2 | 61.8 | 19.5 | 1.23 | 1.13 |
| 90% CI-upper | 66.5 | 21.6 | 82.6 | 24.7 | 1.29 | 1.15 |

In conclusion, PBPK modelling of Formulation I after the administration of multiple inhaled doses predicts that effects of Formulation I on CYP3A4 substrates are minimal. Based on the criteria of the U.S. Food and Drug Administration (FDA) DDI guidance, no clinically significant CYP3A4 DDI was predicted after the administration of Formulation I QD for 14 days.

1. A method of treating a disease or disorder in a subject for whom oral itraconazole is contraindicated, comprising administering to the respiratory tract of the subject a respirable dry powder comprising itraconazole.

2. The method of claim 1, wherein the subject is treated with a second therapeutic agent that is a substrate, inducer, and/or inhibitor of an enzyme or receptor that is inhibited by, or metabolizes, itraconazole.

3. The method of claim 2, wherein the method comprises co-administering itraconazole with the second therapeutic agent to a subject in need thereof.

4. The method of claim 2, wherein the second therapeutic agent is a substrate, inducer, and/or inhibitor of the cytochrome P450 3A4 (CYP3A4) isoenzyme.

5. (canceled)

6. The method of claim 2, wherein the second therapeutic agent is an alpha blocker, a beta blocker, an analgesic, an antiarrhythmic, an antibacterial, an anticoagulant, an antiplatelet drug, an anticonvulsant, an antidiabetic drug, an antihelminthic, an antifungal, an antiprotozoal, an antimigraine drug, an antineoplastic, an antipsychotic, an anxiolytic, a hypnotic, an antiviral, a calcium channel blocker, a cardiovascular drug, a contraceptive, a diuretic, an anticonvulsant, an immunosuppressant, a lipid-lowering drug, a respiratory drug, an antidepressant drug, a urologic drug, a vasopressin receptor antagonist, a nonsteroidal anti-inflammatory drug (NSAID), or a gastrointestinal drug.

7-9. (canceled)

10. The method of claim 3, wherein the itraconazole is administered to the subject less than about 2 days, before or after administering the second therapeutic agent.

11. The method of claim 3, wherein the itraconazole is administered to the subject on the same day as administering the second therapeutic agent.

12. (canceled)

13. The method of claim 1, wherein the respirable dry powder comprises homogenous respirable dry particles that comprise crystalline itraconazole, a stabilizer, a sodium salt, and an excipient.

14. The method of claim 13, wherein the sodium salt is sodium sulfate.

15. The method of claim 13, wherein the itraconazole is a crystalline sub-particle with a Dv50 of about 50 nm to about 5,000 nm.

16. The method of claim 13, wherein the itraconazole is present in the respirable dry particles in an amount of about 30% to about 70% by weight.

17. (canceled)

18. The method of claim 13, wherein the ratio of itraconazole:stabilizer (wt:wt) in the respirable dry particles is about 10:1.

19. (canceled)

20. The method of claim 13, wherein the excipient is present in the respirable dry particles in an amount of about 5% to about 20% by weight or about 10% by weight.

21. The method of claim 13, wherein the stabilizer is polysorbate 80.

22. The method of claim 13, wherein the excipient is leucine.

23. The method of claim 13, wherein the respirable dry powder comprises homogenous respirable dry particles that comprise about 50 wt % crystalline itraconazole, about 35 wt % sodium sulfate, about 10 wt % leucine, and about 5 wt % polysorbate 80.

24. The method of claim 13, wherein the respirable dry particles have:
(i) a volume median geometric diameter (VMGD) of about 10 microns or less;
(ii) a tap density of between 0.2 g/cc and 1.0 g/cc;
(iii) a 1 bar/4 bar dispersibility ratio (¼ bar) of less than about 1.5, as measured by laser diffraction; and/or
(iv) a 0.5 bar/4 bar dispersibility ratio (0.5/4 bar) of about 1.5 or less, as measured by laser diffraction.

25. The method of claim 13, wherein the respirable dry powder has:
(i) a mass median aerodynamic diameter (MMAD) of between about 1 micron and about 5 microns; and/or
(ii) a fine particle fraction (FPF) of the total dose less than 5 microns of about 25% or more.

26. The method of claim 13, wherein the respirable dry particles have a capsule emitted powder mass of at least 80% when emitted from a passive dry powder inhaler that has a resistance of about 0.036 sqrt (kPa)/liters per minute under the following conditions; an inhalation flow rate of 30 LPM for a period of 3 seconds using a size 3 capsule that contains a total mass of 10 mg, said total mass consisting of the respirable dry particles, and wherein the volume median geometric diameter of the respirable dry particles emitted from the inhaler as measured by laser diffraction is 5 microns or less.

27-28. (canceled)

29. The method of claim 1, wherein the disease or disorder is an infection, allergic bronchopulmonary aspergillosis, a respiratory disease, an acute exacerbation of a respiratory disease, or cancer.

30-35. (canceled)
